(19) United States
(12) Patent Application Publication
(10) Pub. No.: US 2026/0062727 A1
(43) Pub. Date: Mar. 5, 2026

(54) METHODS FOR PRODUCING GAMMA-CYCLODEXTRINS

(71) Applicant: BEREN THERAPEUTICS P.B.C., West Hollywood, CA (US)

(72) Inventors: Eli GROBAN, San Francisco, CA (US); Zhihao HU, Jiangsu (CN); Steven PFEIFFER, West Hollywood, CA (US); Benjamin RIZKIN, West Hollywood, CA (US); Gabor BENKOVICS, West Hollywood, CA (US)

(21) Appl. No.: 18/873,273

(22) PCT Filed: Jun. 9, 2023
(86) PCT No.: PCT/IB2023/055979

§ 371 Date: Dec. 9, 2024

(30) Foreign Application Priority Data
Jun. 10, 2022 (WO) PCT/CN2022/098239

(51) Int. Cl. C12P 19/18; C12N 9/10; C12P 19/16

(52) U.S. Cl. CPC C12P 19/18; C12N 9/1074; C12P 19/16; C12Y 204/01004; C12Y 204/01007; C12Y 204/01019

(57) ABSTRACT

Provided herein are methods for the enzymatic production of gamma-cyclodextrin from sucrose. In some cases, the methods involve contacting sucrose with one or more enzymes to convert sucrose to amylose, followed by contacting the amylose with one or more enzymes to convert the amylose to gamma-cyclodextrin. In some cases, the methods produce higher yields of gamma-cyclodextrin relative to alpha-cyclodextrin, beta-cyclodextrin, or both.

### RELATED APPLICATIONS

This application is a United States National Stage Application filed under 35 U.S.C. 371, of International Patent Application No. PCT/IB2023/055979, filed Jun. 9, 2023, which claims priority to International Patent Application No. PCT/CN2022/098239, filed Jun. 10, 2022, the contents of which are incorporated herein by reference in their entirety.

### INCORPORATION BY REFERENCE OF SEQUENCE LISTING

The present application is being filed along with a Sequence Listing in electronic format. The Sequence Listing is provided as a file entitled 061173_522N01US_Sequence Listing, created Oct. 23, 2025, which is 46.7 kilobytes in size. The information in the electronic format of the Sequence Listing is incorporated by reference in its entirety.

### BACKGROUND

Cyclodextrins are a class of cyclic oligosaccharides composed of cyclic oligomers of glucose. Cyclodextrins have a lipophilic central core with hydrophilic outer surfaces, which makes them useful in pharmaceutical and various other industries. The native cyclodextrins (namely alpha-cyclodextrin, beta-cyclodextrin and gamma-cyclodextrin) are designated as generally recognized as safe (GRAS) by the United States Food and Drug Administration (FDA), and are used widely in the food and pharmaceutical industries, among others. Standard methods of producing cyclodextrins generally involve the conversion of starch. However, standard production methods suffer from various disadvantages, including supply chain shortages, scalability, quality variations, purification, and cost of goods. Accordingly, improved methods of producing cyclodextrins, which address these issues, are needed.

### SUMMARY

There is an unmet need for methods for producing cyclodextrins which do not involve the conversion of starch as a starting material. This disclosure meets this unmet need by providing methods for biosynthetically producing cyclodextrins which do not use starch as a starting material. Without being limited to any of the following, advantages of the disclosure provided herein over other methods (e.g., starch-based methods) include higher overall yields of the cyclodextrin product, a more desirable purity, and less byproduct waste, as well as side products, which themselves may be useful for other purposes.

In one aspect, a method of producing a composition comprising cyclodextrin is provided, the method comprising: (a) contacting sucrose with an enzyme, or an enzyme mixture, capable of converting sucrose to amylose under conditions that permit the conversion of the sucrose to amylose, thereby producing amylose; (b) contacting the amylose produced in (a) with an enzyme capable of converting amylose to cyclodextrin under conditions that permit the conversion of the amylose to cyclodextrin, thereby producing the composition comprising cyclodextrin, wherein the composition comprising cyclodextrin comprises gamma-cyclodextrin, and may optionally further comprise alpha-cyclodextrin, beta-cyclodextrin, or any combination thereof, and wherein the composition comprising cyclodextrin comprises gamma-cyclodextrin in an amount and/or concentration greater than alpha-cyclodextrin, beta-cyclodextrin, or both. In some cases, the enzyme of (a) is, or the enzyme mixture of (a) comprises, amylosucrase. In some cases, the amylosucrase is a variant amylosucrase comprising at least one amino acid variant relative to a wild-type amylosucrase. In some cases, the variant amylosucrase is capable of producing a greater amount and/or concentration of amylose from sucrose relative to a wild-type amylosucrase. In some cases, the wild-type amylosucrase is Cellulomonas carboniz T26 amylosucrase. In some cases, the wild-type amylosucrase comprises or consists of the amino acid sequence of SEQ ID NO: 1. In some cases, the wild-type amylosucrase is Neisseria polysaccharea amylosucrase. In some cases, the wild-type amylosucrase comprises or consists of the amino acid sequence of SEQ ID NO: 2. In some cases, the variant amylosucrase comprises or consists of an amino acid sequence having at least about 70% sequence identity to the amino acid sequence of SEQ ID NO: 1 or SEQ ID NO: 2. In some cases, the at least one amino acid variant comprises at least one amino acid substitution relative to a wild-type amylosucrase. In some cases, the at least one amino acid substitution comprises an amino acid substitution at amino acid position 234 relative to a wild-type amylosucrase having the amino acid sequence of SEQ ID NO: 2. In some cases, the amino acid substitution at position 234 is selected from the group consisting of: R234Q, R234G, R234A, R234S, R234M, R234C, R234K, R234I, R234D, R234Y, R234W, R234E, R234L, and R234H. In some cases, the enzyme mixture of (a) comprises at least two enzymes which, collectively or in combination, are capable of converting sucrose to amylose. In some cases, the enzyme mixture comprises sucrose phosphorylase. In some cases, the sucrose phosphorylase is capable of converting sucrose to glucose-1-phosphate. In some cases, the contacting of (a) further comprises contacting the sucrose with the sucrose phosphorylase under conditions that permit the conversion of the sucrose to glucose-1-phosphate. In some cases, the sucrose phosphorylase is selected from the group consisting of: Bifidobacterium longum sucrose phosphorylase, Leuconostoc mesenteroides sucrose phosphorylase, and Streptococcus mutans sucrose phosphorylase. In some cases, the sucrose phosphorylase comprises or consists of the amino acid sequence of any one of SEQ ID NOS: 17-20, or an amino acid sequence having at least about 70% sequence identity to the amino acid sequence of any one of SEQ ID NOs: 17-20. In some cases, the enzyme mixture comprises alpha-glucan phosphorylase. In some cases, the alpha-glucan phosphorylase is capable of converting the glucose-1-phosphate to amylose. In some cases, the contacting of (a) further comprises contacting the glucose-1-phosphate with the alpha-glucan phosphorylase under conditions that permit the conversion of the glucose-1-phosphate to amylose. In some cases, the alpha-glucan phosphorylase is selected from the group consisting of: Solanum tuberosum alpha-glucan phosphorylase, S. tokodaii strain 7 alpha-glucan phosphorylase, and C. callunae DSM 20145 alpha-glucan phosphorylase. In some cases, the alpha-glucan phosphorylase comprises or consists of the amino acid sequence of any one of SEQ ID NOS: 21-24 or an amino acid sequence having at least about 70% sequence identity to the amino acid sequence of any one of SEQ ID NOS: 21-24. In some cases, the enzyme capable of converting the amylose to cyclodextrin in (b) comprises an enzyme capable of producing a greater amount and/or concentration of gamma-cyclodextrin relative to alpha-cyclodextrin, beta-cyclodextrin, or both. In some cases, the enzyme capable of converting the amylose to cyclodextrin in (b) is cyclodextrin glucanotransferase. In some cases, the cyclodextrin glucanotransferase is Bacillus clarkii cyclodextrin glucanotransferase. In some cases, the cyclodextrin glucanotransferase comprises or consists of the amino acid sequence of SEQ ID NO: 25 or 26 or an amino acid sequence having at least about 70% sequence identity to the amino acid sequence of SEQ ID NO: 25 or 26. In some cases, the enzyme capable of converting amylose to cyclodextrin is a variant cyclodextrin glucanotransferase. In some cases, the variant cyclodextrin glucanotransferase is capable of producing a greater amount and/or concentration of gamma-cyclodextrin than alpha-cyclodextrin, beta-cyclodextrin, or both, relative to a wild-type cyclodextrin glucanotransferase. In some cases, the variant cyclodextrin glucanotransferase comprises at least one amino acid variant relative to the wild-type cyclodextrin glucanotransferase. In some cases, the wild-type cyclodextrin glucanotransferase is Bacillus clarkii cyclodextrin glucanotransferase. In some cases, the wild-type cyclodextrin glucanotransferase comprises or consists of the amino acid sequence of SEQ ID NOS: 25 or 26. In some cases, the variant cyclodextrin glucanotransferase comprises or consists of an amino acid sequence having at least about 70% sequence identity to the amino acid sequence of SEQ ID NOS: 25 or 26. In some cases, the at least one amino acid variant comprises at least one amino acid substitution. In some cases, the at least one amino acid substitution comprises an amino acid substitution at amino acid position 186 relative to a wild-type cyclodextrin glucanotransferase having the amino acid sequence of SEQ ID NO: 26. In some cases, the amino acid substitution at position 186 is Y186W. In some cases, the at least one amino acid substitution comprises an amino acid substitution at amino acid position 223 relative to a wild-type cyclodextrin glucanotransferase having the amino acid sequence of SEQ ID NO: 26. In some cases, the amino acid substitution at position 223 is selected from the group consisting of: A223H, A223K, and A223R. In some cases, the contacting of (a) and the contacting of (b) occur sequentially. In some cases, the contacting of (a) and the contacting of (b) occur simultaneously or substantially simultaneously. In some cases, the amylose produced in (a) is not purified or isolated prior to the contacting of (b). In some cases, the contacting of (a), the contacting of (b), or both, is performed in vitro. In some cases, the contacting of (a), the contacting of (b), or both, is performed in a container, a vial, a jar, a test tube, a well, a plate, or an encapsulation. In some cases, the enzyme or at least one enzyme of the enzyme mixture of (a), the variant enzyme of (b), or both, are purified enzymes, isolated enzymes, or both. In some cases, the enzyme or at least one enzyme of the enzyme mixture of (a), the variant enzyme of (b), or both, are recombinantly produced enzymes. In some cases, the contacting of (a), the contacting of (b), or both, is performed in vivo. In some cases, the contacting of (a), the contacting of (b), or both, is performed in a recombinant host cell. In some cases, the recombinant host cell comprises a heterologous nucleic acid encoding the enzyme or at least one enzyme of the enzyme mixture of (a), the variant enzyme of (b), or both. In some cases, the recombinant host cell is a microbial cell. In some cases, the microbial cell is a bacterial cell. In some cases, the enzyme or at least one enzyme of the enzyme mixture of (a), the variant enzyme of (b), or both, is produced in a Pichia yeast cell, such as a Pichia pastoris cell. In some cases, a ratio of gamma-cyclodextrin to alpha-cyclodextrin in the composition comprising cyclodextrin is at least 2:1. In some cases, a ratio of gamma-cyclodextrin to beta-cyclodextrin in the composition comprising cyclodextrin is at least 2:1. In some cases, the composition comprising cyclodextrin comprises no or substantially no alpha-cyclodextrin, beta-cyclodextrin, or both.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C depict the structure of alpha-cyclodextrin, beta-cyclodextrin, and gamma-cyclodextrin, respectively.

FIG. 2A depicts a non-limiting example of a one enzyme reaction to convert sucrose to amylose, in accordance with embodiments of the disclosure.

FIG. 2B depicts a non-limiting example of a two enzyme reaction to convert sucrose to amylose, in accordance with embodiments of the disclosure.

FIG. 3 depicts a non-limiting example of an enzymatic reaction to convert amylose to gamma-cyclodextrin, in accordance with embodiments of the disclosure.

FIG. 4 depicts non-limiting examples of data demonstrating that variant cyclodextrin glucanotransferase enzymes are capable of increasing the ratio of gamma-cyclodextrin produced from amylose relative to alpha-cyclodextrin or beta-cyclodextrin.

FIGS. 5A and 5B depict non-limiting examples of one-pot enzymatic synthesis using variant amylosucrase and variant cyclodextrin glucanotransferase to convert sucrose to gamma-cyclodextrin. FIGS. 5A and 5B show the effect that the reaction time and pH have on the concentration of gamma-cyclodextrin produced.

FIG. 6 depicts graphs of the retained enzymatic activity (%) of freeze-dried whole cell lysate or whole cell slurry of amylosucrase (with or without additives), as discussed in Example 3. The retained enzymatic activity % is measured by comparing the enzymatic activity with whole cell lysate or whole cell slurry which was not freeze-dried.

### DETAILED DESCRIPTION

Current methods of producing cyclodextrins are plagued by supply chain shortages, and issues involving scalability, quality variations, purification, and cost of goods. Additionally, there are several key disadvantages surrounding the current production of cyclodextrins for food and pharmaceutical use, such as, but not limited to, FDA certification of starch after each growing season, and the ability to scale using standard farming techniques. The methods of the present disclosure overcome these issues by providing methods for facile enzymatic synthesis of a composition comprising cyclodextrin from sucrose as a starting material, preferably methods for one-pot enzymatic synthesis.

Provided herein are methods for producing a composition comprising cyclodextrin. Also provided herein are methods for the enzymatic synthesis of gamma-cyclodextrin. Generally, the methods provided herein do not involve the use of starch as a starting material. Preferably, the methods provided herein involve the use of sucrose as a starting material; however, in some embodiments, other mono- or disaccharides may be used. Also provided herein are methods for the enzymatic conversion of sucrose to gamma-cyclodextrin using various enzymes. In various aspects, the methods provided herein generally result in a higher production of gamma-cyclodextrin relative to alpha-cyclodextrin, beta-cyclodextrin, or both. In some cases, the methods provided herein produce higher ratios of gamma-cyclodextrin to alpha-cyclodextrin, gamma-cyclodextrin, or both. The methods generally involve the enzymatic conversion of sucrose to amylose as a first step (step (a)) in the synthesis pathway. In one embodiment, the methods involve the use of a single enzyme, (e.g., amylosucrase), to convert sucrose to amylose. In another embodiment, the methods involve the use of two enzymes, (e.g., sucrose phosphorylase and alpha-glucan phosphorylase), to convert sucrose to amylose. The methods also generally involve the enzymatic conversion of the amylose to gamma-cyclodextrin (e.g., using cyclodextrin glucanotransferase) in a second step (step (b)) in the synthesis pathway. In some embodiments, one or more of the enzymatic steps occurs in vivo (e.g., within a microbial host cell). In some embodiments, one or more of the enzymatic steps occurs in vitro (e.g., in a container, a vial, a jar, a test tube, a well, a plate, an encapsulation, e.g., with purified and/or isolated (e.g., recombinant) enzymes).

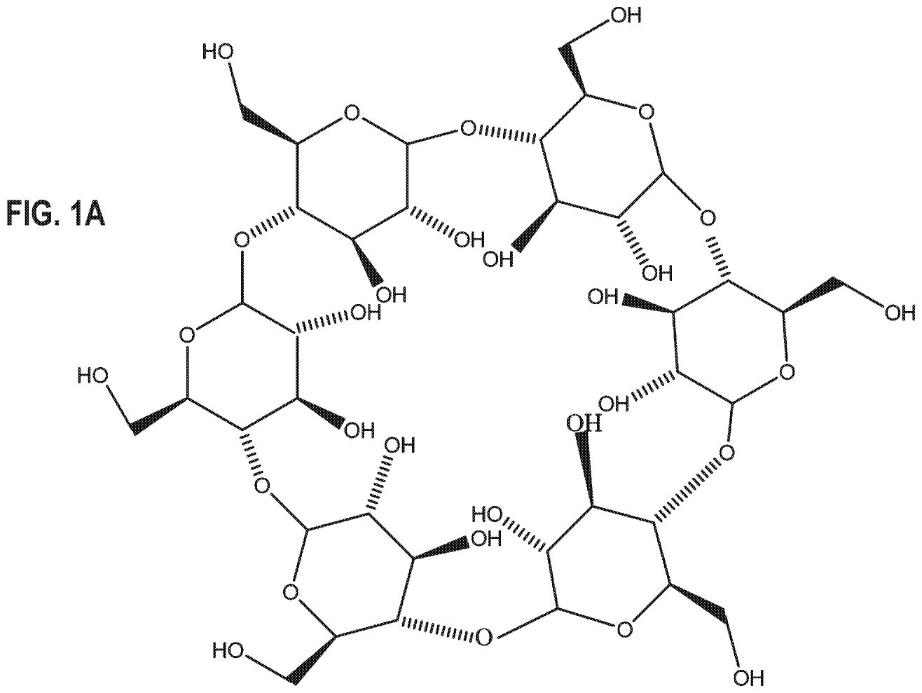

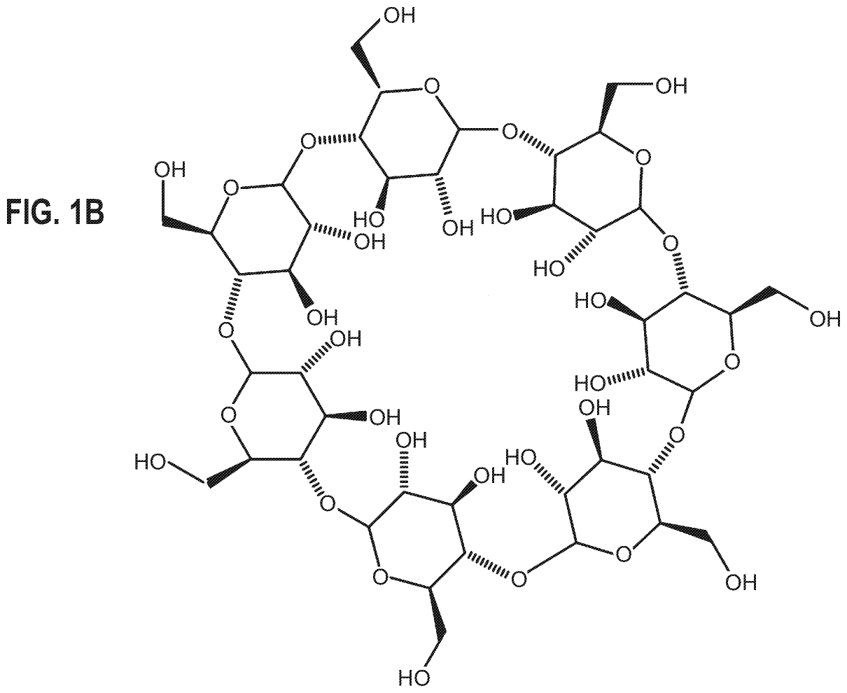

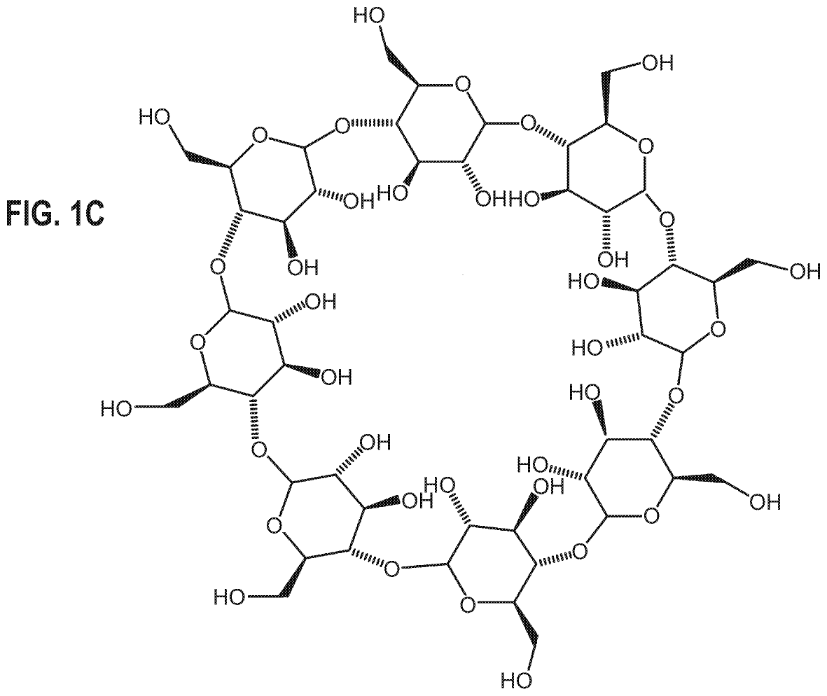

Cyclodextrins are formed by cyclic arrangement of glucopyranose units conjugated by α-1,4 glycosidic linkages. Typically, cyclodextrins are available in three different forms: alpha-cyclodextrin (FIG. 1A), beta-cyclodextrin (FIG. 1B), and gamma-cyclodextrin (FIG. 1C), based on the number of glucose monomers constituting the cyclic arrangement. The number of glucose monomers constituting alpha-cyclodextrin, beta-cyclodextrin, and gamma-cyclodextrin is 6, 7, and 8, respectively. Cyclodextrins have been widely used in food, pharmaceutical, and chemical industries because of their low toxicity, low immunogenicity, and their ability to form noncovalent complexes with guest molecules. For example, cyclodextrins have been widely used as carriers to improve the water solubility of lipophilic vitamins and hormones. In western countries, the ingestion of native cyclodextrins is regulated by the JECFA (Joint WHO/FAO Expert Committee on Food Additives) with the pharmaceutical applications falling under the European Medicines Agency (EMA) in Europe and under the Food and Drug Administration (FDA) in the United States of America. Native cyclodextrins (CDs) can be ingested without significant absorption, being thus ‘Generally Regarded As Safe’ by the FDA, and are commonly referred to as molecules with ‘GRAS status’.

Gamma-cyclodextrins are widely used in the pharmaceutical industry. Different derivatives of gamma-cyclodextrins are fabricated in order to improve the oral bioavailability and solubility of the cyclodextrins. For example, modifying the hydroxyl groups of cyclodextrins with alkyl hydroxyl groups drastically improves the solubility of cyclodextrins. Some of the potential derivatives include replacing all eight primary hydroxy groups with 2-(carboxyethyl) sulfanyl groups or randomly methylated gamma-cyclodextrin and branched gamma-cyclodextrin.

In one aspect of the disclosure, a method of producing a composition comprising cyclodextrin is provided. In some cases, the method comprises (a) contacting sucrose with an enzyme, or an enzyme mixture, capable of converting sucrose to amylose under conditions that permit the conversion of the sucrose to amylose, thereby producing amylose. In some cases, the method further comprises (b) contacting the amylose with an enzyme capable of converting amylose to cyclodextrin under conditions that permit the conversion of the amylose to cyclodextrin, thereby producing the composition comprising cyclodextrin. In some cases, the enzyme capable of converting amylose to cyclodextrin is an enzyme capable of producing a greater amount and/or concentration (e.g., wt %, mol %, or w/v) of gamma-cyclodextrin than alpha-cyclodextrin, beta-cyclodextrin, or both. In some cases, the composition comprising cyclodextrin comprises gamma-cyclodextrin, and may optionally further comprise beta-cyclodextrin, alpha-cyclodextrin, or any combination thereof. In some cases, the composition comprising cyclodextrin comprises gamma-cyclodextrin in an amount and/or concentration (e.g., wt %, mol %, or w/v) greater than alpha-cyclodextrin, beta-cyclodextrin, or both. In some cases, the amount and/or concentration of alpha-cyclodextrin, beta-cyclodextrin, and gamma-cyclodextrin is measured by high-performance liquid chromatography (HPLC).

### Method Step (a) for Enzymatic Conversion of Sucrose to Amylose

The methods provided herein involve the enzymatic conversion of sucrose to amylose. In some embodiments, the methods involve contacting sucrose with an enzyme, or an enzyme mixture, capable of converting sucrose to amylose under conditions that permit the conversion of the sucrose to amylose, thereby producing amylose. In some cases, the amylose is alpha-amylose. In one aspect, the methods involve the use of a single enzyme to convert sucrose to amylose. In alternative aspects, the methods involve the use of an enzyme mixture (e.g., two enzymes), which collectively or in combination, convert sucrose to amylose. In some cases, the sucrose is deuterated sucrose (e.g., one or more hydrogens have been replaced with deuterium). In some cases, the sucrose, and/or any one or more reagents used in the synthesis reaction are deuterated.

One Enzyme Method for Producing Amylose from Sucrose

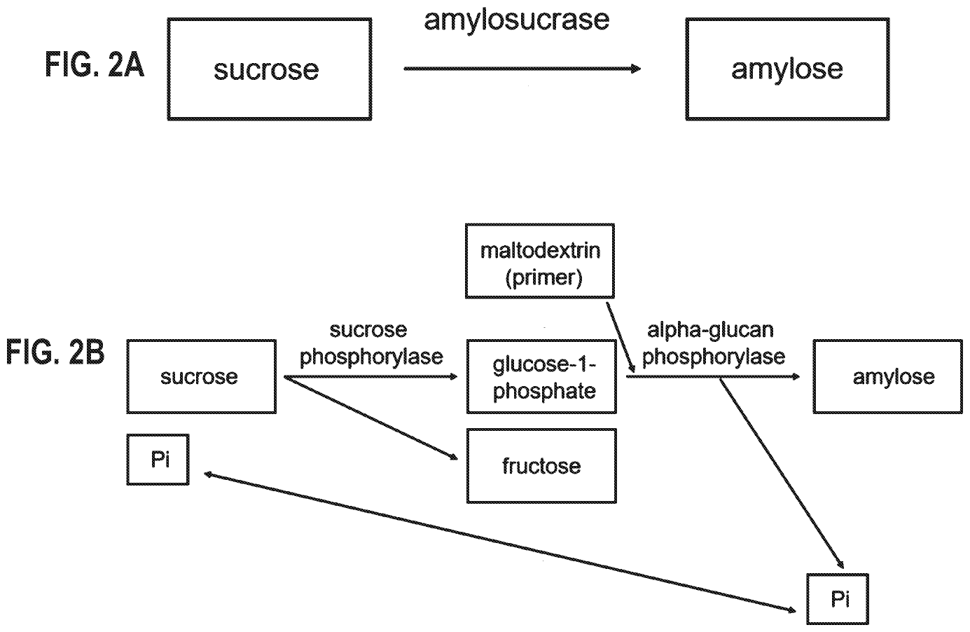

In some aspects, the method for converting sucrose to amylose involves a single enzyme. In some cases, the enzyme is amylosucrase. FIG. 2A depicts a schematic of a single enzyme method of producing amylose from sucrose. In this example, sucrose is contacted with amylosucrase which converts the sucrose to amylose. In some cases, the amylosucrase is a wild-type amylosucrase. For example, the wild-type amylosucrase may be Cellulomonas carboniz T26 amylosucrase (e.g., NCBI Accession No. N868_11335). In some cases, the wild-type Cellulomonas carboniz T26 amylosucrase may comprise or consist of an amino acid sequence according to SEQ ID NO: 1. In some cases, the wild-type amylosucrase may be Neisseria polysaccharea amylosucrase (e.g., NCBI Accession No. AJ011781). In some cases, the wild-type Neisseria polysaccharea amylosucrase may comprise or consist of an amino acid sequence according to SEQ ID NO: 2. Table 1 below depicts non-limiting examples of wild-type amylosucrase enzymes (and their amino acid sequences) that can be used in accordance with the methods provided herein.

TABLE 1
Non-limiting examples of wild-type amylosucrase enzymes

| Enzyme | SEQ ID NO: | Sequence (5′ to 3′) |
|---|---|---|
| Cellulomonas | SEQ ID NO: 1 | MRVSEPLHPSVTAAVAAARDPRTSAEHWAAIEARVAREWPR |
| carboniz T26 | | LERLFLEVYGEGERTRSELAALAHQLVVSAQERPADLRAVD |
| amylosucrase | | VAREADPAWFTSHRMLGGVCYVDRYAGDLEGLRRHIPYLRE |
| | | LGLTYLHLMPLFEAPAENSDGGYAVSSYRRVNPALGTMEQL |
| | | TALAAELRENGISLVLDFIFNHTSDEHEWARRALAGEREYE |
| | | DYYWVFPDREMPDAYERTVREIFPDDHPGSFVPMPDSPDGS |
| | | RTGRWIWATFHSFQWDLNYANPAVFRAMAGEMLFLANKGVD |
| | | VLRMDAVAFIWKQLGTACESLPQAHLLIQAFNAALRIAAPG |
| | | VLFKSEAIVHPDEVVQYISPDECQISYNPLQMALIWSSLAT |
| | | REANLLQQALERRHALPPGTAWVNYVRSHDDIGWTFADEDA |
| | | AELGIDGFQHRRFLNAFYVDRFPGSFARGVPFQDNPRTGDC |
| | | RISGTTASLAGLEARDPGAVDRILLAHSIVLSTGGIPLLYL |
| | | GDEVGQLNDYSYRDQPGLAEDSRWVNRPWYPAQAYANRLVP |
| | | STAAGRVFRGLRHLLEVRRRTPELAGGTLVPFDAHNRHVVG |
| | | YQRPGELDGVPTTVLCLASFADEPQAVEPLTLSGMPAEAED |
| | | LLTGATVDLRAGLVLRPHGFVWLRVRHAA |
| Neisseria | SEQ ID NO: 2 | MLTPTQQVGLILQYLKTRILDIYTPEQRAGIEKSEDWRQFS |
| polysaccharea | | RRMDTHFPKLMNELDSVYGNNEALLPMLEMLLAQAWQSYSQ |
| amylosucrase | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLREIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |

\

In some embodiments, the amylosucrase is a variant amylosucrase comprising at least one amino acid variant relative to a wild-type amylosucrase. The variant amylosucrase may comprise one or more amino acid substitutions, deletions, insertions, and/or modifications relative to a wild-type amylosucrase. In some cases, the variant amylosucrase is capable of producing a greater amount and/or concentration of amylose from sucrose relative to a wild-type amylosucrase.

In some cases, the variant amylosucrase comprises at least one amino acid variant relative to wild-type Cellulomonas carboniz T26 amylosucrase. In some cases, the variant amylosucrase comprises at least one amino acid variant relative to SEQ ID NO: 1. In some cases, the variant amylosucrase comprises at least one amino acid variant relative to wild-type Neisseria polysaccharea amylosucrase. In some cases, the variant amylosucrase comprises at least one amino acid variant relative to SEQ ID NO: 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, relative to wild-type Cellulomonas carboniz T26 amylosucrase. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, relative to the amino acid sequence of SEQ ID NO: 1. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, relative to wild-type Neisseria polysaccharea amylosucrase. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, relative to the amino acid sequence of SEQ ID NO: 2.

In some cases, the at least one variant comprises at least one amino acid substitution relative to a wild-type amylosucrase. In some cases, the at least one amino acid variant comprises at least one amino acid substitution relative to wild-type Cellulomonas carboniz T26 amylosucrase. In some cases, the at least one amino acid variant comprises at least one amino acid substitution relative to wild-type Neisseria polysaccharea amylosucrase. In some cases, the at least one amino acid substitution comprises an amino acid substitution at amino acid position 234 relative to the amino acid sequence of SEQ ID NO: 2. In some cases, the amino acid substitution at amino acid position 234 relative to the amino acid sequence of SEQ ID NO: 2 is selected from the group consisting of: R234Q, R234G, R234A, R234S, R234M, R234C, R234K, R234I, R234D, R234Y, R234W, R234E, R234L, and R234H. In a preferred embodiment, the amino acid substitution at amino acid position 234 relative to the amino acid sequence of SEQ ID NO: 2 is selected from the group consisting of: R234Q, R234G, R234A, R234S, R234M, R234C, and R234K. In this regard, it will be appreciated that R234Q denotes that the arginine (R) at amino acid position 234 relative to the amino acid sequence of SEQ ID NO: 2 is substituted with a glutamine (Q) etc. In some cases, the amino acid substitution at amino acid position 234 relative to the amino acid sequence of SEQ ID NO: 2 is R234Q (e.g., SEQ ID NO: 3 in Table 2). In some cases, the amino acid substitution at amino acid position 234 relative to the amino acid sequence of SEQ ID NO: 2 is R234G (e.g., SEQ ID NO: 4 in Table 2). In some cases, the amino acid substitution at amino acid position 234 relative to the amino acid sequence of SEQ ID NO: 2 is R234A (e.g., SEQ ID NO: 5 in Table 2). In some cases, the amino acid substitution at amino acid position 234 relative to the amino acid sequence of SEQ ID NO: 2 is R234S (e.g., SEQ ID NO: 6 in Table 2). In some cases, the amino acid substitution at amino acid position 234 relative to the amino acid sequence of SEQ ID NO: 2 is R234M (e.g., SEQ ID NO: 7 in Table 2). In some cases, the amino acid substitution at amino acid position 234 relative to the amino acid sequence of SEQ ID NO: 2 is R234C (e.g., SEQ ID NO: 8 in Table 2). In some cases, the amino acid substitution at amino acid position 234 relative to the amino acid sequence of SEQ ID NO: 2 is R234K (e.g., SEQ ID NO: 9 in Table 2). In some cases, the amino acid substitution at amino acid position 234 relative to the amino acid sequence of SEQ ID NO: 2 is R234I (e.g., SEQ ID NO: 10 in Table 2). In some cases, the amino acid substitution at amino acid position 234 relative to the amino acid sequence of SEQ ID NO: 2 is R234D (e.g., SEQ ID NO: 11 in Table 2). In some cases, the amino acid substitution at amino acid position 234 relative to the amino acid sequence of SEQ ID NO: 2 is R234Y (e.g., SEQ ID NO: 12 in Table 2). In some cases, the amino acid substitution at amino acid position 234 relative to the amino acid sequence of SEQ ID NO: 2 is R234W (e.g., SEQ ID NO: 13 in Table 2). In some cases, the amino acid substitution at amino acid position 234 relative to the amino acid sequence of SEQ ID NO: 2 is R234E (e.g., SEQ ID NO: 14 in Table 2). In some cases, the amino acid substitution at amino acid position 234 relative to the amino acid sequence of SEQ ID NO: 2 is R234L (e.g., SEQ ID NO: 15 in Table 2). In some cases, the amino acid substitution at amino acid position 234 relative to the amino acid sequence of SEQ ID NO: 2 is R234H (e.g., SEQ ID NO: 16 in Table 2).

In some aspects, the variant amylosucrase comprises or consists of an amino acid sequence according to any one of SEQ ID NOS: 3-16 or 31, depicted in Table 2, or an amino acid sequence having at least about 70% sequence identity (e.g., at least about 75%, at least about 80%, at least about 85%, at least about 86%, at least about 87%, at least about 88%, at least about 90%, at least about 90%, at least about 91%, at least about 92%, at least about 93%, at least about 94%, at least about 95%, at least about 96%, at least about 97%, at least about 98%, at least about 99%, or greater) to an amino acid sequence according to any one of SEQ ID NOS: 3-16 or 31, depicted in Table 2. In a preferred embodiment, the variant amylosucrase comprises or consists of an amino acid sequence according to any one of SEQ ID NOS: 3-9, depicted in Table 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, as compared to the amino acid sequence of any one of SEQ ID NOS: 3-16, depicted in Table 2. In a preferred embodiment, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, as compared to the amino acid sequence of any one of SEQ ID NOS: 3-9, depicted in Table 2.

TABLE 2
Non-limiting examples of variant amylosucrase enzymes.

| Enzyme | SEQ ID NO: | Sequence (5′ to 3′) |
|---|---|---|
| Amylosucrase | SEQ ID NO: 3 | MLTPTQQVGLILQYLKTRILDIYTPEQRAGIEKSEDWRQFS |
| R234Q | | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLQEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: 4 | MLTPTQQVGLILQYLKTRILDIYTPEQRAGIEKSEDWRQFS |
| R234G | | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLGEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: 5 | MLTPTQQVGLILQYLKTRILDIYTPEQRAGIEKSEDWRQFS |
| R234A | | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLAEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: 6 | MLTPTQQVGLILQYLKTRILDIYTPEQRAGIEKSEDWRQFS |
| R234S | | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLSEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: 7 | MLTPTQQVGLILQYLKTRILDIYTPEQRAGIEKSEDWRQFS |
| R234M | | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLMEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: 8 | MLTPTQQVGLILQYLKTRILDIYTPEQRAGIEKSEDWRQFS |
| R234C | | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLCEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: 9 | MLTPTQQVGLILQYLKTRILDIYTPEQRAGIEKSEDWRQFS |
| R234K | | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLKEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: | MLTPTQQVGLILQYLKTRILDIYTPEQRAGIEKSEDWRQFS |
| R234I | 10 | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLIEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: | MLTPTQQVGLILQYLKTRILDIYTPEQRAGIEKSEDWRQFS |
| R234D | 11 | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLDEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: | MLTPTQQVGLILQYLKTRILDIYTPEQRAGIEKSEDWRQFS |
| R234Y | 12 | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLYEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: | MLTPTQQVGLILQYLKTRILDIYTPEQRAGIEKSEDWRQFS |
| R234W | 13 | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLWEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: | MLTPTQQVGLILQYLKTRILDIYTPEQRAGIEKSEDWRQFS |
| R234E | 14 | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLEEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: | MLTPTQQVGLILQYLKTRILDIYTPEQRAGIEKSEDWRQFS |
| R234L | 15 | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLLEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: | MLTPTQQVGLILQYLKTRILDIYTPEQRAGIEKSEDWRQFS |
| R234H | 16 | RRMDTHFPKLMNELDSVYGNNEALLPMLEMLLAQAWQSYSQ |
| | | RNSSLKDIDIARENNPDWILSNKQVGGVCYVDLFAGDLKGL |
| | | KDKIPYFQELGLTYLHLMPLFKCPEGKSDGGYAVSSYRDVN |
| | | PALGTIGDLREVIAALHEAGISAVVDFIFNHTSNEHEWAQR |
| | | CAAGDPLFDNFYYIFPDRRMPDQYDRTLHEIFPDQHPGGFS |
| | | QLEDGRWVWTTFNSFQWDLNYSNPWVFRAMAGEMLFLANLG |
| | | VDILRMDAVAFIWKQMGTSCENLPQAHALIRAFNAVMRIAA |
| | | PAVFFKSEAIVHPDQVVQYIGQDECQIGYNPLQMALLWNTL |
| | | ATREVNLLHQALTYRHNLPEHTAWVNYVRSHDDIGWTFADE |
| | | DAAYLGISGYDHRQFLNRFFVNRFDGSFARGVPFQYNPSTG |
| | | DCRVSGTAAALVGLAQDDPHAVDRIKLLYSIALSTGGLPLI |
| | | YLGDEVGTLNDDDWSQDSNKSDDSRWAHRPRYNEALYAQRN |
| | | DPSTAAGQIYQGLRHMIAVRQSNPRFDGGRLVTFNTNNKHI |
| | | IGYIRNNALLAFGNFSEYPQTVTAHTLQAMPFKAHDLIGGK |
| | | TVSLNQDLTLQPYQVMWLEIA |
| Amylosucrase | SEQ ID NO: | MGLILQYLKTRILDIYTPEQRAGIEKSEDWRQFSRRMDTHF |
| R234Ndelta8A | 31 | PKLMNELDSVYGNNEALLPMLEMLLAQAWQSYSQRNSSLKD |
| A | | IDIARENNPDWILSNKQVGGVCYVDLFAGDLKGLKDKIPYF |
| | | QELGLTYLHLMPLFKCPEGKSDGGYAVSSYRDVNPALGTIG |
| | | DLREVIAALHEAGISAVVDFIFNHTSNEHEWAQRCAAGDPL |
| | | FDNFYYIFPDRRMPDQYDRTLNEIFPDQHPGGFSQLEDGRW |
| | | VWTTFNSFQWDLNYSNPWVFRAMAGEMLFLANLGVDILRMD |
| | | AVAFIWKQMGTSCENLPQAHALIRAFNAVMRIAAPAVFFKS |
| | | EAIVHPDQVVQYIGQDECQIGYNPLQMALLWNTLATREVNL |
| | | LHQALTYRHNLPEHTAWVNYVRSHDDIGWTFADEDAAYLGI |
| | | SGYDHRQFLNRFFVNRFDGSFARGVPFQYNPSTGDCRVSGT |
| | | AAALVGLAQDDPHAVDRIKLLYSIALSTGGLPLIYLGDEVG |
| | | TLNDDDWSQDSNKSDDSRWAHRPRYNEALYAQRNDPSTAAG |
| | | QIYQGLRHMIAVRQSNPRFDGGRLVTFNTNNKHIIGYIRNN |
| | | ALLAFGNFSEYPQTVTAHTLQAMPFKAHDLIGGKTVSLNQD |
| | | LTLQPYQVMWLEIA |

In some aspects, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and an amino acid substitution at amino acid position 234 relative to SEQ ID NO: 2. In this regard, and as used throughout the disclosure, the stated sequence identity includes the specified amino acid substitution (i.e., the sequence identity is calculated based on the entire amino acid sequence of the variant enzyme, including the specified amino acid substitution). In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and an amino acid substitution at amino acid position 234 relative to SEQ ID NO: 2 selected from the group consisting of: R234Q, R234G, R234A, R234S, R234M, R234C, R234K, R234I, R234D, R234Y, R234W, R234E, R234L, and R234H. In a preferred embodiment, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and an amino acid substitution at amino acid position 234 relative to SEQ ID NO: 2 selected from the group consisting of: R234Q, R234G, R234A, R234S, R234M, R234C, and R234K. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or NO: 2, and the amino acid substitution R234Q relative to SEQ ID NO: 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and the amino acid substitution R234G relative to SEQ ID NO: 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and the amino acid substitution R234A relative to SEQ ID NO: 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and the amino acid substitution R234S relative to SEQ ID NO: 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and the amino acid substitution R234M relative to SEQ ID NO: 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or NO: 2, and the amino acid substitution R234C relative to SEQ ID NO: 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and the amino acid substitution R234K relative to SEQ ID NO: 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and the amino acid substitution R234I relative to SEQ ID NO: 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and the amino acid substitution R234D relative to SEQ ID NO: 2. In some cases, the variant amylosucrase comprises or consists an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and the amino acid substitution R234Y relative to SEQ ID NO: 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or NO: 2, and the amino acid substitution R234W relative to SEQ ID NO: 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and the amino acid substitution R234E relative to SEQ ID NO: 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and the amino acid substitution R234L relative to SEQ ID NO: 2. In some cases, the variant amylosucrase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 2, and the amino acid substitution R234H relative to SEQ ID NO: 2.

In some embodiments, the amylosucrase is derived from a microbial cell. In some cases, the amylosucrase is isolated and/or purified from a microbial cell. In some cases, the microbial cell is a bacterial cell. In some cases, the bacterial cell is Escherichia coli. In some embodiments, the amylosucrase is derived from Neisseria polysaccharea. In some embodiments, the amylosucrase is derived from Cellulomonas carboniz T26. In some embodiments, the amylosucrase may be produced within a microbial cell. In some embodiments, the amylosucrase is expressed in a recombinant host cell (e.g., from a recombinant polynucleotide). In some cases, the amylosucrase is recombinantly produced. In some cases, the amylosucrase is produced (e.g., recombinantly produced) in a yeast cell. In some cases, the yeast cell is a Pichia yeast cell, such as a Pichia pastoris cell.

Two Enzyme Method for Producing Amylose from Sucrose

In some aspects, the methods involve contacting sucrose with an enzyme mixture capable of converting sucrose to amylose under conditions that permit the conversion of the sucrose to amylose, thereby producing amylose. In some cases, the methods involve contacting sucrose with an enzyme mixture that contains at least two enzymes, which, collectively or in combination, are capable of converting the sucrose to amylose. For example, the enzyme mixture may contain at least sucrose phosphorylase and alpha-glucan phosphorylase. The methods may involve contacting sucrose with the at least two enzymes simultaneously or substantially simultaneously. Alternatively, the methods may involve contacting sucrose with the at least two enzymes sequentially. FIG. 2B depicts a schematic of a two enzyme method of producing amylose from sucrose. In this example, sucrose is contacted with sucrose phosphorylase to convert the sucrose to glucose-1-phosphate. The glucose-1-phosphate is then contacted with alpha-glucan phosphorylase to convert the glucose-1-phosphate to amylose. In some cases, the sucrose phosphorylase and the alpha-glucan phosphorylase are contacted with the sucrose simultaneously or substantially simultaneously. In other cases, the sucrose phosphorylase and the alpha-glucan phosphorylase are added sequentially (e.g., the sucrose phosphorylase is contacted with the sucrose first to generate glucose-1-phosphate, then the alpha-glucan phosphorylase is added to generate the amylose). In some cases, the glucose-1-phosphate generated from the reaction with sucrose phosphorylase is isolated and/or purified prior to contacting the glucose-1-phosphate with the alpha-glucan phosphorylase. In other cases, the glucose-1-phosphate generated from the reaction with sucrose phosphorylase is not isolated and/or purified prior to contacting the glucose-1-phosphate with the alpha-glucan phosphorylase. The term “substantially simultaneously” when used in context with the addition of two or more components to a reaction mixture as described herein means the two or more components are added to the reaction mixture within 10 seconds or less of one another.

In some cases, the sucrose phosphorylase is a wild-type sucrose phosphorylase. For example, the wild-type sucrose phosphorylase may be Bifidobacterium longum sucrose phosphorylase (e.g., NCBI Accession No. AAO84039). In some cases, the wild-type Bifidobacterium longum sucrose phosphorylase may comprises or consist of the amino acid sequence according to SEQ ID NO: 17. In some cases, the wild-type sucrose phosphorylase may be Leuconostoc mesenteroide sucrose phosphorylase (e.g., NCBI Accession No. D90314.1). In some cases, the wild-type Leuconostoc mesenteroide sucrose phosphorylase may comprise or consist of the amino acid sequence according to SEQ ID NO: 18. In some cases, the wild-type sucrose phosphorylase may be Streptococcus mutans sucrose phosphorylase (e.g., NCBI Accession No. NZ_CP013237.1). In some cases, the wild-type Streptococcus mutans sucrose phosphorylase may comprise or consist of the amino acid sequence according to SEQ ID NO: 19 (e.g., NCBI Accession No. P10249). In some cases, the sucrose phosphorylase enzyme is a variant sucrose phosphorylase enzyme. In some cases, the variant sucrose phosphorylase has one or more amino acid substitutions relative to a wild-type sucrose phosphorylase. In some cases, the variant sucrose phosphorylase has an amino acid substitution at one or more of, or all of, amino acid residues T47, S62, Y77, V128, K140, Q144, N155, and D249, relative to SEQ ID NO: 19. In some cases, the amino acid substitution at amino acid position 47 relative to SEQ ID NO: 19 is T47S. In some cases, the amino acid substitution at amino acid position 62 relative to SEQ ID NO: 19 is S62P. In some cases, the amino acid substitution at amino acid position 77 relative to SEQ ID NO: 19 is Y77H. In some cases, the amino acid substitution at amino acid position 128 relative to SEQ ID NO: 19 is V128L. In some cases, the amino acid substitution at amino acid position 140 relative to SEQ ID NO: 19 is K140M. In some cases, the amino acid substitution at amino acid position 144 relative to SEQ ID NO: 19 is Q144R. In some cases, the amino acid substitution at amino acid position 155 relative to SEQ ID NO: 19 is N155S. In some cases, the amino acid substitution at amino acid position 249 relative to SEQ ID NO: 19 is D249G. In some cases, the variant sucrose phosphorylase has amino acid substitutions T47S, S62P, Y77H, V128L, K140M, Q144R, N155S, and D249G, relative to SEQ ID NO: 19. In some cases, the variant sucrose phosphorylase enzyme comprises or consists of the amino acid sequence according to SEQ ID NO: 20. Table 3 below depicts non-limiting examples of sucrose phosphorylase enzymes (and their amino acid sequences) that can be used in accordance with the methods provided herein.

TABLE 3
Non-limiting examples of sucrose phosphorylase enzymes

| Enzyme | SEQ ID NO: | Sequence (5′ to 3′) |
|---|---|---|
| Bifidobacterium | SEQ ID NO: | MKNKVQLITYADRLGDGTLSSMADILRTRFDGVYDGVHILP |
| longum sucrose | 17 | FFTPFDGADAGFDPIDHTKVDERLGSWDDVAELSKTHNIMV |
| phosphorylase | | DAIVNHMSWESKQFQDVLEKGEESEYYPMFLTMSSVFPNGA |
| | | TEEDLAGIYRPRPGLPFTHYKFAGKTRLVWVSFTPQQVDIDT |
| | | DSDKGWEYLMSIFDQMAASHVSYIRLDAVGYGAKEAGTSCF |
| | | MTPKTFKLISRLREEGVKRGLEILIEVHSYYKKQVEIASKVDR |
| | | VYDFALPPLLLHSLFTGHVEPVAHWTEIRPNNAVTVLDTHD |
| | | GIGVIDIGSDQLDRSLKGLVPDEDVDNLVNTIHANTHGESQA |
| | | ATGAAASNLDLYQVNSTYYSALGCNDQHYLAARAVQFFLP |
| | | GVPQVYYVGALAGRNDMELLRRTNNGRDINRHYYSTAEIDE |
| | | NLERPVVKALNALAKFRNELPAFDGEFSYEVDGDTSITFRWT |
| | | AADGTSTAALTFEPGRGLGTDNATPVASLAWSDAAGDHETR |
| | | DLLANPPIADID |
| Leuconostoc | SEQ ID NO: | MEIQNKAMLITYADSLGKNLKDVHQVLKEDIGDAIGGVHLL |
| mesenteroides | 18 | PFFPSTGDRGFAPADYTRVDAAFGDWADVEALGEEYYLMF |
| sucrose | | DFMINHISRESVMYQDFKKNHDDSKYKDFFIRWEKFWAKAG |
| phosphorylase | | ENRPTQADVDLIYKRKDKAPTQEITFDDGTTENLWNTFGEEQ |
| | | IDIDVNSAIAKEFIKTTLEDMVKHGANLIRLDAFAYAVKKVD |
| | | TNDFFVEPEIWDTLNEVREILTPLKAEILPEIHEHYSIPKKIND |
| | | HGYFTYDFALPMTTLYTLYSGKTNQLAKWLKMSPMKQFTT |
| | | LDTHDGIGVVDARDILTDDEIDYASEQLYKVGANVKKTYSS |
| | | ASYNNLDIYQINSTYYSALGNDDAAYLLSRVFQVFAPGIPQIY |
| | | YVGLLAGENDIALLESTKEGRNINRHYYTREEVKSEVKRPVV |
| | | ANLLKLLSWRNESPAFDLAGSITVDTPTDTTIVVTRQDENGQ |
| | | NKAVLTADAANKTFEIVENGQTVMSSDNLTQN |
| Streptococcus | SEQ ID NO: | MPIINKTMLITYADSLGKNLKELNENIENYFGDAVGGVHLLP |
| mutans sucrose | 19 | FFPSTGDRGFAPIDYHEVDSAFGDWDDVKCLGEKYYLMFDF |
| phosphorylase | | MINHISRQSKYYKDYQEKHEASAYKDLFLNWDKFWPKNRP |
| | | TQEDVDLIYKRKDRAPKQEIQFADGSVEHLWNTFGEEQIDLD |
| | | VTKEVTMDFIRSTIENLAANGCDLIRLDAFAYAVKKLDTNDF |
| | | FVEPEIWTLLDKVRDIAAVSGAEILPEIHEHYTIQFKIADHDY |
| | | YVYDFALPMVTLYSLYSSKVDRLAKWLKMSPMKQFTTLDT |
| | | HDGIGVVDVKDILTDEEITYTSNELYKVGANVNRKYSTAEY |
| | | NNLDIYQINSTYYSALGDDDQKYFLARLIQAFAPGIPQVYYV |
| | | GFLAGKNDLELLESTKEGRNINRHYYSSEEIAKEVKRPVVKA |
| | | LLNLFTYRNQSAAFDLDGRIEVETPNEATIVIERQNKDGSHIA |
| | | KAEINLQDMTYRVTENDQTISFE |
| SP3-M8 (T47S, | SEQ ID NO: | MPITNKTMLITYADSLGKNLKELNENIENYFGDAVGGVHLLP |
| S62P, Y77H, | 20 | FFPSSGDRGFAPIDYHEVDPAFGDWDDVKRLGEKHYLMFDF |
| V128L, K140M, | | MINHISRQSKYYKDYQEKHEASAYKDLFLNWDKFWPKNRP |
| Q144R, N155S | | TQEDLDLIYKRKDRAPMQEIRFADGSVEHLWSTFGEEQIDLD |
| and D249G) | | VTKEVTMDFIRSTIENLAANGCDLIRLDAFAYAVKKLDTNDF |
| | | FVEPEIWTLLDKVRDIAAVSGAEILPEIHEHYTIQFKIADHGY |
| | | YVYDFALPMVTLYSLYSGKVDRLAKWLKMSPMKQFTTLDT |
| | | HDGIGVVDVKDILTDEEITYTSNELYKVGANVNRKYSTAEY |
| | | NNLDIYQINSTYYSALGDDDQKYFLARLIQAFAPGIPQVYYV |
| | | GFLAGKNDLELLESTKEGRNINRHYYSSEEIAKEVKRPVVKA |
| | | LLNLFTYRNQSAAFDLDGRIEVETPNEATIVIERQNKDGSHIA |
| | | TAEINLQDMTYRVTENDQTISFE |

In some cases, the sucrose phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to wild-type Bifidobacterium longum sucrose phosphorylase. In some cases, the sucrose phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 17. In some cases, the sucrose phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to wild-type Leuconostoc mesenteroides sucrose phosphorylase. In some cases, the sucrose phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 18. In some cases, the sucrose phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to wild-type Streptococcus mutans sucrose phosphorylase. In some cases, the sucrose phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 19. In some cases, the sucrose phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 20, and comprises the amino acid substitutions T47S, S62P, Y77H, V128L, K140M, Q144R, N155S, and D249G, relative to SEQ ID NO: 19.

In some embodiments, the sucrose phosphorylase is derived from a microbial cell. In some cases, the sucrose phosphorylase is isolated and/or purified from a microbial cell. In some cases, the microbial cell is a bacterial cell. In some cases, the bacterial cell is Escherichia coli. In some embodiments, the sucrose phosphorylase is derived from Bifidobacterium longum. In some embodiments, the sucrose phosphorylase is derived from Leuconostoc mesenteroides. In some embodiments, the sucrose phosphorylase is derived from Streptococcus mutans. In some embodiments, the sucrose phosphorylase may be produced within a microbial cell. In some embodiments, the sucrose phosphorylase is expressed in a recombinant host cell (e.g., from a recombinant polynucleotide). In some cases, the sucrose phosphorylase is recombinantly produced. In some cases, the sucrose phosphorylase is produced (e.g., recombinantly produced) in a yeast cell. In some cases, the yeast cell is a Pichia yeast cell, such as a Pichia pastoris cell.

In some aspects, the alpha-glucan phosphorylase is a wild-type alpha-glucan phosphorylase. In some cases, the wild-type alpha-glucan phosphorylase may be Solanum tuberosum alpha-glucan phosphorylase (e.g., NCBI Accession No. D00520.1). In some cases, the wild-type Solanum tuberosum alpha-glucan phosphorylase may comprise or consists of the amino acid sequence according to SEQ ID NO: 21. In some cases, the wild-type alpha-glucan phosphorylase may be S. tokodaii strain 7 alpha-glucan phosphorylase (e.g., NCBI Accession No. NC_003106.2). In some cases, the wild-type S. tokodaii strain 7 alpha-glucan phosphorylase may comprise or consists of the amino acid sequence according to SEQ ID NO: 22. In some cases, the wild-type alpha-glucan phosphorylase may be C. callunae DSM 20145 alpha-glucan phosphorylase (e.g., NCBI Accession No. AY102616.1). In some cases, the wild-type C. callunae DSM 20145 alpha-glucan phosphorylase may comprise or consist of the amino acid sequence according to SEQ ID NO: 23. In some cases, the alpha-glucan phosphorylase enzyme is a variant alpha-glucan phosphorylase enzyme. In some cases, the variant alpha-glucan phosphorylase has one or more amino acid substitutions relative to a wild-type alpha-glucan phosphorylase. In some cases, the variant alpha-glucan phosphorylase has an amino acid substitution at one or more of, or all of, amino acid residues F39, N135, and T706, relative to SEQ ID NO: 21. In some cases, the amino acid substitution at amino acid position 39 relative to SEQ ID NO: 21 is F39L. In some cases, the amino acid substitution at amino acid position 135 relative to SEQ ID NO: 21 is N135S. In some cases, the amino acid substitution at amino acid position 706 relative to SEQ ID NO: 21 is T706I. In some cases, the variant alpha-glucan phosphorylase has amino acid substitutions F39L, N135S, and T706I, relative to SEQ ID NO: 21. In some cases, the variant alpha-glucan phosphorylase enzyme comprises or consists of the amino acid sequence according to SEQ ID NO: 24. Table 4 below depicts non-limiting examples of alpha-glucan phosphorylase enzymes (and their amino acid sequences) that can be used in accordance with the methods provided herein.

TABLE 4
Non-limiting examples of alpha-glucan phosphorylase enzymes

| | SEQ ID NO: | Sequence (5′ to 3′) |
|---|---|---|
| Solanum | SEQ ID NO: | TLSEKIHHPITEQGGESDLSSFAPDAASITSSIKYHAEFTPVFSP |
| tuberosum | 21 | ERFELPKAFFATAQSVRDSLLINWNATYDIYEKLNMKQAYY |
| alpha-glucan | | LSMEFLQGRALLNAIGNLELTGAFAEALKNLGHNLENVASQ |
| phosphorylase | | EPDAALGNGGLGRLASCFLDSLATLNYPAWGYGLRYKYGLF |
| | | KQRITKDGQEEVAEDWLEIGSPWEVVRNDVSYPIKFYGKVS |
| | | TGSDGKRYWIGGEDIKAVAYDVPIPGYKTRTTISLRLWSTQV |
| | | PSADFDLSAFNAGEHTKACEAQANAEKICYILYPGDESEEGK |
| | | ILRLKQQYTLCSASLQDIISRFERRSGDRIKWEEFPEKVAVQM |
| | | NDTHPTLCIPELMRILIDLKGLNWNEAWNITQRTVAYTNHTV |
| | | LPEALEKWSYELMQKLLPRHVEIIEAIDEELVHEIVLKYGSM |
| | | DLNKLEEKLTTMRILENFDLPSSVAELFIKPEISVDDDTETVE |
| | | VHDKVEASDKVVINDEDDTGKKTSVKIEAAAEKDIDKKTPV |
| | | SPEPAVIPPKKVRMANLCVVGGHAVNGVAEIHSEIVKEEVEN |
| | | DFYELWPEKFQNKTNGVTPRRWIRFCNPPLSAIITKWTGTED |
| | | WVLKTEKLAELQKFADNEDLQNEWREAKRSNKIKVVSFLKE |
| | | KTGYSVVPDAMFDIQVKRIHEYKRQLLNIFGIVYRYKKMKE |
| | | MTAAERKTNFVPRVCIFGGKAFATYVQAKRIVKFITDVGATI |
| | | NHDPEIGDLLKVVFVPDYNVSVAELLIPASDLSEHISTAGME |
| | | ASGTSNMKFAMNGCIQIGTLDGANVEIREEVGEENFFLFGAQ |
| | | AHEIAGLRKERADGKFVPDERFEEVKEFVRSGAFGSYNYDD |
| | | LIGSLEGNEGFGRADYFLVGKDFPSYIECQEKVDEAYRDQKR |
| | | WTTMSILNTAGSYKFSSDRTIHEYAKDIWNIEAVEIA |
| S. tokodaii | SEQ ID NO: | MRKHLKGKAHKKLHMIISITAELGIDFGENFAGGLGVLEGDK |
| strain 7 alpha- | 22 | FYASARLGIDYTVFTLFYRKGYTGNEEKQKELLKNLVKEWE |
| glucan | | TEIELKKGKIKIEYLTYKLNTAKAIFINILSPDWAKRLNEKLYI |
| phosphorylase | | ENSEEDRFYKYLVLAKATEKYISEKIGWDKIKYVDLQEAYPS |
| | | FLPLLKYFPRYRIIIHTPAPWGHPTFPARYFKEEFGFEFPFDPV |
| | | VMTEIGLSSAVQGIVVSKKMLHHVSKTFPHHMHKIKAITNA |
| | | VEIPRWRHPLLNNVKDLDDFIKKKKEVKKESLKKLGKESDK |
| | | PTIGWVRRITQYKRPEFILRLIDELRDDVVFIIGGKAHPYEYY |
| | | GVELEKKFKEYAQKRNNVIYVQGVDIQQMKLAIWSSDIWTF |
| | | TPYSGWEASGTSFMKAGVNGVPSVASRDGAVPEIIKDGYNG |
| | | WLYGEDRYELLPVDTYDREYEEFARKVKEALNKYYEVGYN |
| | | AYHTFSDFCSMDRLMKEYAL |
| C. callunae | SEQ ID NO: | MSPEKQPLPAALVGSHVRAAAGTPADLATDRKFWTGLSRA |
| DSM 20145 | 23 | VQERIADDWERTREAYGAARQQHYFSAEFLMGRALLNNLT |
| alpha-glucan | | NLGLVDEAAAATRELGHELTDILEIENDAALGNGGLGRLAA |
| phosphorylase | | CFLDSAVTQDYPVTGYGLLYRFGLFRQSFNEGFQVEKPDPW |
| | | REEEYPFTIRRASDQLVVCFDDMKTRAIPYDMPITGYGTHNV |
| | | GTLRLWKAEPWEEFDYDAFNSQRFTDAHIERERVSDICRVLY |
| | | PNDTTYEGKKLRVRQQYFFTSASLQAMIQDHLAHHKDLSNF |
| | | AEFHSVQLNDTHPVLAIPELMRLLMDEHDMGWEESWAIVSK |
| | | TFAYTNHTVLTEALEQWDEQIFQQLFWRVWEIIAEIDRRFRL |
| | | ERAADGLDEETINRMAPIQHGTVHMAWIACYAAYSINGVAA |
| | | LHTEIIKAETLADWYALWPEKFNNKTNGVTPRRWLRMINPG |
| | | LSDLLTRLSGSDDWVTDLDELKKLRSYADDKSVLEELRAIK |
| | | AANKQDFAEWILERQGIEIDPESIFDVQIKRLHEYKRQLMNA |
| | | LYVLDLYFRIKEDGLTDIPARTVIFGAKAAPGYVRAKAIIKLI |
| | | NSIADLVNNDPEVSPLLKVVFVENYNVSPAEHILPASDVSEQI |
| | | STAGKEASGTSNMKFMMNGALTLGTMDGANVEIVDSVGEE |
| | | NAYIFGARVEELPALRESYKPYELYETVPGLKRALDALDNGT |
| | | LNDNNSGLFYDLKHSLIHGYGKDASDTYYVLGDFADYRETR |
| | | DRMAADYASDPLGWARMAWINICESGRFSSDRTIRDYATEI |
| | | WKLEPTPAVKK |
| GP-M3 (GP | SEQ ID NO: | TLSEKIHHPITEQGGESDLSSFAPDAASITSSIKYHAELTPVFSP |
| F39L N135S | 24 | ERFELPKAFFATAQSVRDSLLINWNATYDIYEKLNMKQAYY |
| T706I) | | LSMEFLQGRALLNAIGNLELTGAFAEALKNLGHNLENVASQ |
| | | EPDAALGSGGLGRLASCFLDSLATLNYPAWGYGLRYKYGLF |
| | | KQRITKDGQEEVAEDWLEIGSPWEVVRNDVSYPIKFYGKVS |
| | | TGSDGKRYWIGGEDIKAVAYDVPIPGYKTRTTISLRLWSTQV |
| | | PSADFDLSAFNAGEHTKACEAQANAEKICYILYPGDESEEGK |
| | | ILRLKQQYTLCSASLQDIISRFERRSGDRIKWEEFPEKVAVQM |
| | | NDTHPTLCIPELMRILIDLKGLNWNEAWNITQRTVAYTNHTV |
| | | LPEALEKWSYELMQKLLPRHVEIIEAIDEELVHEIVLKYGSM |
| | | DLNKLEEKLTTMRILENFDLPSSVAELFIKPEISVDDDTETVE |
| | | VHDKVEASDKVVINDEDDTGKKTSVKIEAAAEKDIDKKTPV |
| | | SPEPAVIPPKKVRMANLCVVGGHAVNGVAEIHSEIVKEEVEN |
| | | DFYELWPEKFQNKTNGVTPRRWIRFCNPPLSAIITKWTGTED |
| | | WVLKTEKLAELQKFADNEDLQNEWREAKRSNKIKVVSFLKE |
| | | KTGYSVVPDAMFDIQVKRIHEYKRQLLNIFGIVYRYKKMKE |
| | | MTAAERKTNFVPRVCIFGGKAFATYVQAKRIVKFIIDVGATI |
| | | NHDPEIGDLLKVVFVPDYNVSVAELLIPASDLSEHISTAGME |
| | | ASGTSNMKFAMNGCIQIGTLDGANVEIREEVGEENFFLFGAQ |
| | | AHEIAGLRKERADGKFVPDERFEEVKEFVRSGAFGSYNYDD |
| | | LIGSLEGNEGFGRADYFLVGKDFPSYIECQEKVDEAYRDQKR |
| | | WTTMSILNTAGSYKFSSDRTIHEYAKDIWNIEAVEIA |

In some cases, the alpha-glucan phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to wild-type Solanum tuberosum alpha-glucan phosphorylase. In some cases, the alpha-glucan phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 21. In some cases, the alpha-glucan phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to wild-type S. tokodaii strain 7 alpha-glucan phosphorylase. In some cases, the alpha-glucan phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 22. In some cases, the alpha-glucan phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to wild-type C. callunae DSM 20145 alpha-glucan phosphorylase. In some cases, the alpha-glucan phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 23. In some cases, the sucrose phosphorylase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 21, and comprises the amino acid substitutions F39L, N135S, and T706I, relative to SEQ ID NO: 21.

In some embodiments, the alpha-glucan phosphorylase is derived from a microbial cell. In some cases, the alpha-glucan phosphorylase is isolated and/or purified from a microbial cell. In some cases, the microbial cell is a bacterial cell. In some cases, the bacterial cell is Escherichia coli. In some embodiments, the alpha-glucan phosphorylase is derived from Solanum tuberosum. In some embodiments, the alpha-glucan phosphorylase is derived from S. tokodaii strain 7. In some embodiments, the alpha-glucan phosphorylase is derived from C. callunae DSM 20145. In some embodiments, the alpha-glucan phosphorylase may be produced within a microbial cell. In some embodiments, the alpha-glucan phosphorylase is expressed in a recombinant host cell (e.g., from a recombinant polynucleotide). In some cases, the alpha-glucan phosphorylase is recombinantly produced. In some cases, the alpha-phosphorylase is produced (e.g., recombinantly produced) in a yeast cell. In some cases, the yeast cell is a Pichia yeast cell, such as a Pichia pastoris cell.

### Method Step (b) for Enzymatic Conversion of Amylose to Gamma-Cyclodextrin

In various aspects, the methods further comprise enzymatically converting the amylose (e.g., produced by the methods (e.g., method step (a)) provided herein) to cyclodextrin, preferably gamma-cyclodextrin. In some cases, the methods comprise contacting the amylose with an enzyme capable of converting amylose to cyclodextrin under conditions that permit the conversion of the amylose to cyclodextrin. In some cases, the enzyme capable of converting amylose to cyclodextrin is an enzyme capable of producing a greater amount and/or concentration of gamma-cyclodextrin than alpha-cyclodextrin, beta-cyclodextrin, or both.

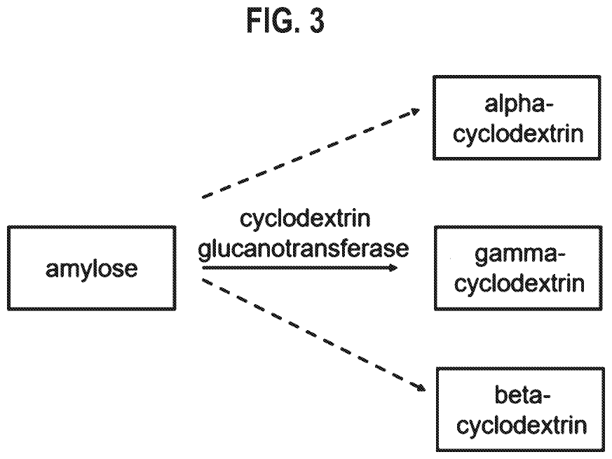

In some aspects, the enzyme capable of converting the amylose to cyclodextrin comprises a cyclodextrin glucanotransferase. FIG. 3 depicts the enzymatic conversion of amylose to gamma-cyclodextrin with cyclodextrin glucanotransferase. Preferably, the gamma-cyclodextrin glucanotransferase produces gamma-cyclodextrin from amylose in an amount and/or concentration greater than an amount and/or concentration of alpha-cyclodextrin and/or beta-cyclodextrin. In some cases, the cyclodextrin glucanotransferase is a wild-type cyclodextrin glucanotransferase. In some cases, the wild-type cyclodextrin glucanotransferase is Bacillus clarkii cyclodextrin glucanotransferase (e.g., NCBI Accession No. AB432985.1). In some cases, the wild-type cyclodextrin glucanotransferase comprises or consists of an amino acid sequence according to SEQ ID NO: 25 or 26, or an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence according to SEQ ID NO: 25 or 26.

In some cases, the cyclodextrin glucanotransferase is a variant cyclodextrin glucanotransferase. In some cases, the variant cyclodextrin glucanotransferase comprises at least one amino acid variant relative to a wild-type cyclodextrin glucanotransferase. The variant cyclodextrin glucanotransferase may comprise one or more amino acid substitutions, deletions, insertions, and/or modifications relative to a wild-type cyclodextrin glucanotransferase. In some cases, the variant cyclodextrin glucanotransferase is capable of producing a greater amount and/or concentration of cyclodextrin relative to alpha-cyclodextrin and/or beta-cyclodextrin from amylose relative to a wild-type cyclodextrin glucanotransferase.

In some cases, the variant cyclodextrin glucanotransferase comprises at least one amino acid variant relative to wild-type Bacillus clarkii cyclodextrin glucanotransferase (e.g., NCBI Accession No. AB432985.1; e.g., SEQ ID NO: 25 or SEQ ID NO: 26). In some cases, the variant cyclodextrin glucanotransferase comprises at least one amino acid variant relative to SEQ ID NO: 25 or SEQ ID NO: 26. In some cases, the variant cyclodextrin glucanotransferase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of wild-type Bacillus clarkii cyclodextrin glucanotransferase. In some cases, the variant cyclodextrin glucanotransferase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 25 or SEQ ID NO: 26.

In some cases, the at least one amino acid variant comprises at least one amino acid substitution relative to a wild-type cyclodextrin glucanotransferase. In some cases, the at least one amino acid substitution comprises an amino acid substitution at amino acid position 186 relative to the amino acid sequence of SEQ ID NO: 26. In some cases, the amino acid substitution at amino acid position 186 relative to the amino acid sequence of SEQ ID NO: 26 is Y186W (e.g., SEQ ID NO: 27 in Table 5). In some cases, the at least one amino acid substitution comprises an amino acid substitution at amino acid position 223 relative to the amino acid sequence of SEQ ID NO: 26. In some cases, the amino acid substitution at amino acid position 223 relative to the amino acid sequence of SEQ ID NO: 26 is A223H (e.g., SEQ ID NO: 28 in Table 5). In some cases, the amino acid substitution at amino acid position 223 relative to the amino acid sequence of SEQ ID NO: 26 is A223K (e.g., SEQ ID NO: 29 in Table 5). In some cases, the amino acid substitution at amino acid position 223 relative to the amino acid sequence of SEQ ID NO: 26 is A223R (e.g., SEQ ID NO: 30 in Table 5).

In some aspects, the cyclodextrin glucanotransferase comprises or consists of an amino acid sequence according to any one of SEQ ID NOS: 25-30, depicted in Table 5. In some aspects, the cyclodextrin glucanotransferase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of any one of SEQ ID NOS: 25-30, depicted in Table 5.

TABLE 5
Non-limiting examples of cyclodextrin glucanotransferase enzymes

| | SEQ ID NO: | Sequence (5′ to 3′) |
|---|---|---|
| Wild-type | SEQ ID NO: | MFRKLLCTLVTIITLSAWIVSHGGEVHASNATNDLSNVNYAE |
| Bacillus clarkii | 25 | EVIYHIVTDRFKDGDPDNNPQGQLFSNGCSDLTKYCGGDWQ |
| cyclodextrin | | GIIDEIESGYLPDMGITALWISPPVENVFDLHPEGFSSYHGYW |
| glucanotransfer | | ARDFKKTNPFFGDFDDFSRLIETAHAHDIKVVIDFVPNHTSPV |
| ase (BKcgt) | | DIEDGALYDNGTLLGHYSTDANNYFYNYGGSDFSDYENSIY |
| | | RNLYDLASLNQQHSFIDKYLKESIQLWLDTGIDGIRVDAVAH |
| | | MPLGWQKAFISSVYDYNPVFTFGEWFTGAQGSNHYHHFVN |
| | | NSGMSALDFRYAQVAQDVLRNQKGTMHDIYDMLASTQLDY |
| | | ERPQDQVTFIDNHDIDRFTVEGRDTRTTDIGLAFLLTSRGVPA |
| | | IYYGTENYMTGKGDPGNRKMMESFDQTTTAYQVIQKLAPLR |
| | | QENKAVAYGSTKERWINDDVLIYERSFNGDYLLVAINKNVN |
| | | QAYTISGLLTEMPAQVYHDVLDSLLDGQSLAVKENGTVDSF |
| | | LLGPGEVSVWQHISESGSAPVIGQVGPPMGKPGDAVKISGSG |
| | | FGSEPGTVYFRDTKIDVLTWDDETIVITLPETLGGKAQISVTN |
| | | SDGVTSNGYDFQLLTGKQESVRFVVDNAHTNYGENVYLVG |
| | | NVPELGNWNPADAIGPMFNQVVYSYPTWYYDVSVPADTAL |
| | | EFKFIIVDGNGNVTWESGGNHNYRVTSGSTDTVRVSFRR |
| Wild-type | SEQ ID NO: | SNATNDLSNVNYAEEVIYHIVTDRFKDGDPDNNPQGQLFSN |
| Bacillus clarkii | 26 | GCSDLTKYCGGDWQGIIDEIESGYLPDMGITALWISPPVENVF |
| cyclodextrin | | DLHPEGFSSYHGYWARDFKKTNPFFGDFDDFSRLIETAHAHD |
| glucanotransferase | | IKVVIDFVPNHTSPVDIEDGALYDNGTLLGHYSTDANNYFYN |
| (BKcgt) | | YGGSDFSDYENSIYRNLYDLASLNQQHSFIDKYLKESIQLWL |
| mature chain | | DTGIDGIRVDAVAHMPLGWQKAFISSVYDYNPVFTFGEWFT |
| | | GAQGSNHYHHFVNNSGMSALDFRYAQVAQDVLRNQKGTM |
| | | HDIYDMLASTQLDYERPQDQVTFIDNHDIDRFTVEGRDTRTT |
| | | DIGLAFLLTSRGVPAIYYGTENYMTGKGDPGNRKMMESFDQ |
| | | TTTAYQVIQKLAPLRQENKAVAYGSTKERWINDDVLIYERSF |
| | | NGDYLLVAINKNVNQAYTISGLLTEMPAQVYHDVLDSLLDG |
| | | QSLAVKENGTVDSFLLGPGEVSVWQHISESGSAPVIGQVGPP |
| | | MGKPGDAVKISGSGFGSEPGTVYFRDTKIDVLTWDDETIVIT |
| | | LPETLGGKAQISVTNSDGVTSNGYDFQLLTGKQESVRFVVD |
| | | NAHTNYGENVYLVGNVPELGNWNPADAIGPMFNQVVYSYP |
| | | TWYYDVSVPADTALEFKFIIVDGNGNVTWESGGNHNYRVTS |
| | | GSTDTVRVSFRR |
| BKcgt Y186W | SEQ ID NO: | SNATNDLSNVNYAEEVIYHIVTDRFKDGDPDNNPQGQLFSN |
| | 27 | GCSDLTKYCGGDWQGIIDEIESGYLPDMGITALWISPPVENVF |
| | | DLHPEGFSSYHGYWARDFKKTNPFFGDFDDFSRLIETAHAHD |
| | | IKVVIDFVPNHTSPVDIEDGALYDNGTLLGHYSTDANNYFYN |
| | | YGGSDFSDYENSIYRNLWDLASLNQQHSFIDKYLKESIQLWL |
| | | DTGIDGIRVDAVAHMPLGWQKAFISSVYDYNPVFTFGEWFT |
| | | GAQGSNHYHHFVNNSGMSALDFRYAQVAQDVLRNQKGTM |
| | | HDIYDMLASTQLDYERPQDQVTFIDNHDIDRFTVEGRDTRTT |
| | | DIGLAFLLTSRGVPAIYYGTENYMTGKGDPGNRKMMESFDQ |
| | | TTTAYQVIQKLAPLRQENKAVAYGSTKERWINDDVLIYERSF |
| | | NGDYLLVAINKNVNQAYTISGLLTEMPAQVYHDVLDSLLDG |
| | | QSLAVKENGTVDSFLLGPGEVSVWQHISESGSAPVIGQVGPP |
| | | MGKPGDAVKISGSGFGSEPGTVYFRDTKIDVLTWDDETIVIT |
| | | LPETLGGKAQISVTNSDGVTSNGYDFQLLTGKQESVRFVVD |
| | | NAHTNYGENVYLVGNVPELGNWNPADAIGPMFNQVVYSYP |
| | | TWYYDVSVPADTALEFKFIIVDGNGNVTWESGGNHNYRVTS |
| | | GSTDTVRVSFRR |
| BKcgt A223H | SEQ ID NO: | SNATNDLSNVNYAEEVIYHIVTDRFKDGDPDNNPQGQLFSN |
| | 28 | GCSDLTKYCGGDWQGIIDEIESGYLPDMGITALWISPPVENVF |
| | | DLHPEGFSSYHGYWARDFKKTNPFFGDFDDFSRLIETAHAHD |
| | | IKVVIDFVPNHTSPVDIEDGALYDNGTLLGHYSTDANNYFYN |
| | | YGGSDFSDYENSIYRNLYDLASLNQQHSFIDKYLKESIQLWL |
| | | DTGIDGIRVDAVHHMPLGWQKAFISSVYDYNPVFTFGEWFT |
| | | GAQGSNHYHHFVNNSGMSALDFRYAQVAQDVLRNQKGTM |
| | | HDIYDMLASTQLDYERPQDQVTFIDNHDIDRFTVEGRDTRTT |
| | | DIGLAFLLTSRGVPAIYYGTENYMTGKGDPGNRKMMESFDQ |
| | | TTTAYQVIQKLAPLRQENKAVAYGSTKERWINDDVLIYERSF |
| | | NGDYLLVAINKNVNQAYTISGLLTEMPAQVYHDVLDSLLDG |
| | | QSLAVKENGTVDSFLLGPGEVSVWQHISESGSAPVIGQVGPP |
| | | MGKPGDAVKISGSGFGSEPGTVYFRDTKIDVLTWDDETIVIT |
| | | LPETLGGKAQISVTNSDGVTSNGYDFQLLTGKQESVRFVVD |
| | | NAHTNYGENVYLVGNVPELGNWNPADAIGPMFNQVVYSYP |
| | | TWYYDVSVPADTALEFKFIIVDGNGNVTWESGGNHNYRVTS |
| | | GSTDTVRVSFRR |
| BKcgt A223K | SEQ ID NO: | SNATNDLSNVNYAEEVIYHIVTDRFKDGDPDNNPQGQLFSN |
| | 29 | GCSDLTKYCGGDWQGIIDEIESGYLPDMGITALWISPPVENVF |
| | | DLHPEGFSSYHGYWARDFKKTNPFFGDFDDFSRLIETAHAHD |
| | | IKVVIDFVPNHTSPVDIEDGALYDNGTLLGHYSTDANNYFYN |
| | | YGGSDFSDYENSIYRNLYDLASLNQQHSFIDKYLKESIQLWL |
| | | DTGIDGIRVDAVKHMPLGWQKAFISSVYDYNPVFTFGEWFT |
| | | GAQGSNHYHHFVNNSGMSALDFRYAQVAQDVLRNQKGTM |
| | | HDIYDMLASTQLDYERPQDQVTFIDNHDIDRFTVEGRDTRTT |
| | | DIGLAFLLTSRGVPAIYYGTENYMTGKGDPGNRKMMESFDQ |
| | | TTTAYQVIQKLAPLRQENKAVAYGSTKERWINDDVLIYERSF |
| | | NGDYLLVAINKNVNQAYTISGLLTEMPAQVYHDVLDSLLDG |
| | | QSLAVKENGTVDSFLLGPGEVSVWQHISESGSAPVIGQVGPP |
| | | MGKPGDAVKISGSGFGSEPGTVYFRDTKIDVLTWDDETIVIT |
| | | LPETLGGKAQISVTNSDGVTSNGYDFQLLTGKQESVRFVVD |
| | | NAHTNYGENVYLVGNVPELGNWNPADAIGPMFNQVVYSYP |
| | | TWYYDVSVPADTALEFKFIIVDGNGNVTWESGGNHNYRVTS |
| | | GSTDTVRVSFRR |
| BKcgt A223R | SEQ ID NO: | SNATNDLSNVNYAEEVIYHIVTDRFKDGDPDNNPQGQLFSN |
| | 30 | GCSDLTKYCGGDWQGIIDEIESGYLPDMGITALWISPPVENVF |
| | | DLHPEGFSSYHGYWARDFKKTNPFFGDFDDFSRLIETAHAHD |
| | | IKVVIDFVPNHTSPVDIEDGALYDNGTLLGHYSTDANNYFYN |
| | | YGGSDFSDYENSIYRNLYDLASLNQQHSFIDKYLKESIQLWL |
| | | DTGIDGIRVDAVRHMPLGWQKAFISSVYDYNPVFTFGEWFT |
| | | GAQGSNHYHHFVNNSGMSALDFRYAQVAQDVLRNQKGTM |
| | | HDIYDMLASTQLDYERPQDQVTFIDNHDIDRFTVEGRDTRTT |
| | | DIGLAFLLTSRGVPAIYYGTENYMTGKGDPGNRKMMESFDQ |
| | | TTTAYQVIQKLAPLRQENKAVAYGSTKERWINDDVLIYERSF |
| | | NGDYLLVAINKNVNQAYTISGLLTEMPAQVYHDVLDSLLDG |
| | | QSLAVKENGTVDSFLLGPGEVSVWQHISESGSAPVIGQVGPP |
| | | MGKPGDAVKISGSGFGSEPGTVYFRDTKIDVLTWDDETIVIT |
| | | LPETLGGKAQISVTNSDGVTSNGYDFQLLTGKQESVRFVVD |
| | | NAHTNYGENVYLVGNVPELGNWNPADAIGPMFNQVVYSYP |
| | | TWYYDVSVPADTALEFKFIIVDGNGNVTWESGGNHNYRVTS |
| | | GSTDTVRVSFRR |

In some aspects, the variant cyclodextrin glucanotransferase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 26, and an amino acid substitution at amino acid position 186 relative to SEQ ID NO: 26. In some cases, the variant cyclodextrin glucanotransferase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 26, and the amino acid substitution Y186W relative to SEQ ID NO: 26.

In some aspects, the variant cyclodextrin glucanotransferase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 26, and an amino acid substitution at amino acid position 223 relative to SEQ ID NO: 26. In some cases, the variant cyclodextrin glucanotransferase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 26, and the amino acid substitution A223H relative to SEQ ID NO: 26. In some cases, the variant cyclodextrin glucanotransferase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 26, and the amino acid substitution A223K relative to SEQ ID NO: 26. In some cases, the variant cyclodextrin glucanotransferase comprises or consists of an amino acid sequence having at least about 70% sequence identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater), preferably at least about 90% sequence identity, to the amino acid sequence of SEQ ID NO: 26, and the amino acid substitution A223R relative to SEQ ID NO: 26.

In some embodiments, the cyclodextrin glucanotransferase is derived from a microbial cell. In some cases, the cyclodextrin glucanotransferase is isolated and/or purified from a microbial cell. In some cases, the microbial cell is a bacterial cell. In some cases, the bacterial cell is Escherichia coli. In some embodiments, the cyclodextrin glucanotransferase is derived from Bacillus clarkii. In some embodiments, the cyclodextrin glucanotransferase may be produced within a microbial cell. In some embodiments, the cyclodextrin glucanotransferase is expressed in a recombinant host cell (e.g., from a recombinant polynucleotide). In some cases, the cyclodextrin glucanotransferase is recombinantly produced. In some cases, the cyclodextrin glucanotransferase is produced (e.g., recombinantly produced) in a yeast cell. In some cases, the yeast cell is a Pichia yeast cell, such as a Pichia pastoris cell.

In various aspects, the methods provided herein produce a higher ratio of gamma-cyclodextrin to alpha-cyclodextrin, beta-cyclodextrin, or both. For example, in some cases, the methods provided herein provide ratios of gamma-cyclodextrin to alpha-cyclodextrin, beta-cyclodextrin, or both, of at least 2:1, at least 3:1, at least 4:1, at least 5:1, at least 6:1, at least 7:1, at least 8:1, at least 9:1, at least 10:1, at least 20:1, at least 30:1, at least 40:1, at least 50:1, at least 60:1, at least 70:1, at least 80:1, at least 90:1, at least 100:1, or greater. In a preferred embodiment, the methods provided herein provide ratios of gamma-cyclodextrin to alpha-cyclodextrin of at least 10:1. For example, the ratios may be at least 20:1, at least 30:1, at least 40:1, at least 50:1, at least 60:1, at least 70:1, at least 80:1, at least 90:1, at least 100:1, or greater. In a preferred embodiment, the methods provided herein provide ratios of gamma-cyclodextrin to beta-cyclodextrin of at least 3:1. For example, the ratios may be at least 5:1, at least 10:1, at least 20:1, at least 30:1, at least 40:1, at least 50:1, at least 60:1, at least 70:1, at least 80:1, at least 90:1, at least 100:1, or greater. In a preferred embodiment, the methods provided herein provide ratios of beta-cyclodextrin to both alpha-cyclodextrin and gamma-cyclodextrin of at least 3:1. For example, the ratios may be at least 5:1, at least 10:1, at least 20:1, at least 30:1, at least 40:1, at least 50:1, at least 60:1, at least 70:1, at least 80:1, at least 90:1, at least 100:1, or greater.

Methods are outlined throughout the disclosure for attaining robust enzyme activity in each step to obtain higher yields of gamma-cyclodextrin than is currently achievable. In some embodiments, the first enzymatic step of converting sucrose to amylose (e.g., as described herein) is carried out for a first time period, thereby enabling catalytic conversion of sucrose to amylose, followed by the second enzymatic step of converting the amylose to gamma-cyclodextrin (e.g., as described herein), which is carried out for a second time period, thereby enabling catalytic conversion of amylose to gamma-cyclodextrin. In some embodiments, the first enzymatic reaction (e.g., converting sucrose to amylose, e.g., as described herein) and the second enzymatic reaction (e.g., converting amylose to gamma-cyclodextrin, e.g., as described herein) are carried out in the same reservoir (e.g., one-pot synthesis method).

In some embodiments, the first time period is at least 30 minutes, at least 45 minutes, at least 60 minutes, at least 85 minutes, at least 90 minutes, at least 105 minutes, at least 120 minutes, at least 135 minutes, at least 150 minutes, at least 165 minutes, at least 180 minutes, at least 195 minutes, at least 210 minutes, at least 225 minutes, at least 240 minutes, at least 255 minutes, at least 270 minutes, at least 285 minutes, or at least 300 minutes. In some embodiments, the second time period is at least 30 minutes, at least 45 minutes, at least 60 minutes, at least 85 minutes, at least 90 minutes, at least 105 minutes, at least 120 minutes, at least 135 minutes, at least 150 minutes, at least 165 minutes, at least 180 minutes, at least 195 minutes, at least 210 minutes, at least 225 minutes, at least 240 minutes, at least 255 minutes, at least 270 minutes, at least 285 minutes, or at least 300 minutes. In some embodiments, the first time period is shorter than the second time period. In some embodiments, the first time period is longer than the second time period. In some embodiments, the first time period is the same or substantially the same length as the second time period. In some embodiments, sucrose is added to the reaction reservoir in batches. In some embodiments, the enzymes used in the first enzymatic reaction step (e.g., to convert sucrose to amylose, e.g., as described herein) are added once at the beginning of the reaction period and then again after a period of time has elapsed to expedite the catalytic activity. In some embodiments, sucrose is added once at the beginning of the reaction period and then again after a period of time has elapsed to replenish the sucrose. In some embodiments, the enzymes involved in the first enzymatic reaction step (e.g., to convert sucrose to amylose, e.g., as described herein) are added at the same time as the enzymes involved in the second enzymatic reaction step (e.g., to convert amylose to gamma-cyclodextrin) in the same reaction reservoir. In some embodiments, the enzymes involved in the first enzymatic reaction step (e.g., to convert sucrose to amylose, e.g., as described herein) are added at a different time (e.g., before) than the enzymes involved in the second enzymatic reaction step (e.g., to convert amylose to gamma-cyclodextrin).

In some embodiments, the sucrose concentration is maximized for highly efficient conversion to amylose. In some embodiments, the starting concentration of sucrose in the reaction is at least about 50 g/L. In some embodiments, the starting concentration of sucrose in the reaction is at least about 100 g/L. In some embodiments, the starting concentration of sucrose in the reaction is at least about 150 g/L. In some embodiments, the starting concentration of sucrose in the reaction is at least about 200 g/L. In some embodiments, the starting concentration of sucrose in the reaction is at least about 250 g/L. In some embodiments, the starting concentration of sucrose in the reaction is at least about 300 g/L. In some embodiments, the starting concentration of sucrose in the reaction is at least about 350 g/L.

In some embodiments, the reaction time is an important consideration for obtaining maximum yield of gamma-cyclodextrin. In some embodiments, production of gamma-cyclodextrin may be accompanied by breakdown of the product to glucose, maltose, and other sugars. It is therefore important to obtain gamma-cyclodextrin without allowing its breakdown. In some embodiments, the total (e.g., method step (a) and method step (b)) reaction is carried out for no more than 12 hours. In some embodiments, the total (e.g., method step (a) and method step (b)) reaction is carried out for no more than 8 hours. In some embodiments, the total reaction is carried out for no more than 7 hours. In some embodiments, the total reaction is carried out for no more than 6 hours. In some embodiments, the total reaction is carried out for no more than 5 hours. In some embodiments, the total reaction is carried out for no more than 4 hours. In some embodiments, the total reaction is carried out for no more than 3 hours. In some embodiments, the total reaction is carried out for no more than 2 hours. In some embodiments, the total reaction is carried out for no more than 1 hour.

Temperature is an important consideration for maximizing the yield of gamma-cyclodextrin. In some embodiments, one or more of the enzymatic reactions is carried out at from about 30° C. to about 55° C., such as from about 40° C. to about 50° C. In some embodiments, one or more of the enzymatic reactions is carried out at about 40° C. In some embodiments, one or more of the enzymatic reactions is carried out at about 41° C. In some embodiments, one or more of the enzymatic reactions is carried out at about 42° C. In some embodiments, one or more of the enzymatic reactions is carried out at about 43° C. In some embodiments, one or more of the enzymatic reactions is carried out at about 44° C. In some embodiments, one or more of the enzymatic reactions is carried out at about 45° C. In some embodiments, one or more of the enzymatic reactions is carried out at about 46° C. In some embodiments, one or more of the enzymatic reactions is carried out at about 47° C. In some embodiments, one or more of the enzymatic reactions is carried out at about 48° C. In some embodiments, one or more of the enzymatic reactions is carried out at about 49° C. In some embodiments, one or more of the enzymatic reactions is carried out at about 50° C. Preferably, one or more of the reactions is carried out at about 45° C., at about 50° C. or at about 55° C.

In some embodiments, the enzymatic reaction of step (a) is carried out at from about 40° C. to about 55° C., such as from about 45° C. to about 50° C. In some embodiments, the enzymatic reaction of step (b) is carried out at from about 40° C. to about 50° C. Step (a) and step (b) may be carried out at different temperatures or, preferably, step (a) and step (b) are carried out at about the same temperature. For example, the enzymatic reaction of step (a) is preferably carried out at about 50° C. or about 55° C. In this embodiment, the enzymatic reaction of step (b) is preferably also carried out at about 50° C. or about 55° C. respectively.

In a one-pot synthesis, it is taken into consideration that the enzyme mixture(s) should be maximally functional even though the optimum temperature for each enzyme may be slightly different.

In some embodiments, the reaction is carried out at a pH of from 5.0 to 7.5, such as from 6.0 to 7.0, such as about 7.0. In some embodiments, the reaction is carried out at a pH of 6.0. In some embodiments, the reaction is carried out at a pH of 7.0.

In some embodiments, one or more of the enzymatic reactions is carried out in a reaction mixture comprising a buffer. Any suitable buffer known in the art may be used. For example, the buffer may be selected from the group consisting of sodium citrate, disodium hydrogen phosphate, and Tris-HCl. The buffer may be present in the mixture at a concentration of from about 50 mM to about 200 mM. In a preferred embodiment, the buffer is present at a concentration of about 100 mM.

In some embodiments, the reaction is carried out in a reservoir having a reservoir volume of from about 1 mL to about 1,000,000 L. For example, the reaction may be carried out in a reservoir having a reservoir volume of from about 100 mL to about 10 L, such as a reservoir volume of about 500 mL or about 10 L.

In some embodiments, the total reaction volume is from about 1 mL to about 1,000,000 L. For example, the total reaction volume may be from about 100 mL to about 10 L, such as a total reaction volume of about 500 mL or about 5 L. In some embodiments, the total reaction volume is less than the reservoir volume. For example, a total reaction volume of about 5 L may be used in a reaction carried out in a reservoir having a reservoir volume of about 10 L.

In some embodiments, the reaction is carried out in a stirred tank reactor (STR), a loop reactor, a plug flow reactor, a single or multi-stage continuous stirred tank reactor, or any other suitable reactor known in the art. In some embodiments, the reaction is carried out in a stirred tank reactor, wherein the reaction is stirred at from about 100 to about 200 rpm, such as about 160 rpm.

In some embodiments, one or more of the enzymatic reactions is carried out in a reaction mixture comprising an organic solvent, preferably toluene. The reaction mixture preferably also comprises water. Without wishing to be bound by any theory set out herein, the inventors have identified that addition of the organic solvent surprisingly increases the yield of gamma-cyclodextrin obtained from the enzymatic reactions. For example, the addition of the organic solvent may increase the yield of the gamma-cyclodextrin by at least about 5%, for example by at least about 10%, for example by at least about 15%, for example by at least about 20%, for example by at least about 50%, for example by at least about 100%, for example by at least about 150%, for example by at least about 200%, for example by at least about 250%, for example by at least about 300%, for example by at least about 350%, for example by at least about 400% compared to the yield obtained from the enzymatic reactions carried out without the organic solvent. It is believed that the addition of the organic solvent increases the yield of the gamma-cyclodextrin by decreasing the solubility of the gamma-cyclodextrin in the reaction mixture, thereby causing the gamma-cyclodextrin to precipitate, which reduces the concentration of gamma-cyclodextrin in the reaction mixture. This prevents breakdown of the gamma-cyclodextrin by the enzymes.

In some embodiments, the amount of the organic solvent (preferably toluene) in the reaction mixture is from about 0.1% to about 40% v/v of the reaction mixture, such as from about 1% to about 35% v/v, such as from about 5% to about 25% v/v.

In some embodiments, the organic solvent is introduced at the start, or during, the enzymatic reaction of step (a). In some preferred embodiments, the organic solvent is introduced at the start, or during, the enzymatic reaction of step (b). For example, in embodiments where the total (e.g., method step (a) and method step (b)) reaction is carried out for no more than 8 hours, the organic solvent may be introduced about 1 hour after the start of enzymatic reaction (b).

In some embodiments, the enzyme used in step (a) is amylosucrase. In some embodiments, the starting concentration of amylosucrase in the reaction mixture is from about 1 to about 30 U/mL, for example from about 5 to about 25 U/mL, for example from about 8 to about 25 U/mL.

In some embodiments, the enzyme mixture used in step (a) comprises sucrose phosphorylase and alpha-glucan phosphorylase. In some embodiments, the starting concentration of sucrose phosphorylase in the reaction mixture is from about 1 to about 30 U/mL, for example from about 5 to about 25 U/mL, for example from about 8 to about 25 U/mL. In some embodiments, the starting concentration of alpha-glucan phosphorylase in the reaction mixture is from about 1 to about 30 U/mL, for example from about 5 to about 25 U/mL, for example from about 8 to about 25 U/mL.

In some embodiments, the enzymes are provided in whole cell lysate, preferably wherein the ratio of the starting concentrations (measured as volume of whole cell lysate) of enzymes in step (b) to the enzymes in step (a) is from about 1:1 to about 50:1, such as from about 2:1 to about 50:1. such as from about 5:1 to about 40:1, such as from about 10:1 to about 30:1. In a preferred embodiment, the ratio is about 20:1.

In certain embodiments, any one of the enzymatic reactions provided herein (e.g., the first enzymatic reaction to convert sucrose to amylose and/or the second enzymatic reaction to convert amylose to gamma-cyclodextrin) may take place within a microbial host cell. For example, the microbial host cell may comprise one or more heterologous nucleic acid molecules that encode for one or more the enzymes provided herein. The microbial host cell may express one or more of the enzymes provided herein. In some cases, the microbial host cell can be fed sucrose and/or one or more intermediates of the enzymatic reaction. For example, sucrose may be fed to the microbial host cell, and the conversion of sucrose to gamma-cyclodextrin may occur within the microbial host cell.

In some embodiments, one or more of the enzymes used in the enzymatic reactions provided herein may be immobilized on a resin. For example, the enzymes may be covalently linked to a resin. Alternatively, the enzymes may be non-covalently linked to the resin. For example, the enzymes may be linked to a Ni-resin via a His-tag. For example, the enzyme of (a) may be a variant amylosucrase (for example wherein the variant amylosucrase may comprise or consist of an amino acid sequence according to SEQ ID NO: 3) and the enzyme may be immobilized on a resin. Alternatively, or additionally, the enzyme of (b) may be a variant cyclodextrin glucanotransferase and the enzyme may be immobilized on a resin. Optionally, the enzyme or enzyme mixture of (a) and the enzyme of (b) are immobilized on the same resin.

The immobilized resin enzymes may be re-used in the methods described herein. However, the present inventors have found that the gamma-cyclodextrin yield tends to decrease when the immobilized resin enzymes are re-used, which is believed to be due to the enzyme leaching from the resin during use which results in a lower enzymatic conversion. It would therefore be desirable to improve the enzyme stability on the resin and hence prevent enzyme leaching, because this would allow the immobilized resin enzymes to be re-used more often and/or with a higher rate of enzymatic conversion, thereby increasing the yield of the reaction.

The present inventors have found that the enzyme stability may be improved by using freeze-dried enzymes, by spray drying the enzymes, and/or by introducing additives.

In some embodiments, the enzymes are provided in a cell slurry or in whole cell lysate. For example, a cell slurry comprising recombinant cells expressing the enzymes may be suspended in buffer (such as sodium citrate buffer), lysed, and centrifuged to provide a whole cell lysate comprising the enzymes. Methods of cell lysis are known in the art. For examples, the cells may be lysed by homogenization, chemical lysis, sonication, freeze/thaw, lytic enzymes, acidic lysis, and/or alkaline lysis. In a preferred embodiment, the cells are lysed by homogenization.

In some embodiments, the cell slurry or whole cell lysate further comprises an additive. In some embodiments, the additive is selected from the group consisting of PEG, maltose, sorbitol, sucrose, glucose, mannitol, lactose, milk powder, starch and combinations thereof. In some embodiments, the additive is added in an amount from about 0.1% w/v to about 10% w/v of the cell slurry or whole cell lysate, for example from about 0.5% w/v to about 5% w/v. For example, the additive may be added at 0.5% w/v, 1.0% w/v, or 5% w/v of the cell slurry or whole cell lysate. In a preferred embodiment, the additive is mannitol, sorbitol, sucrose, or a combination thereof.

In some embodiments, the cell slurry or cell lysate may be freeze-dried. For example, cell slurry or cell lysate may be freeze-dried over 2 days. Methods of freeze-drying are known in the art.

The inventors have found that the addition of the additive to the cell slurry or whole cell lysate (as described above) increases the enzyme stability compared to a cell slurry or whole cell lysate which does not contain the additive, and that freeze-drying the cell slurry or whole cell lysate (as described above) increases the enzyme stability compared to a cell slurry or whole cell lysate which has not been freeze-dried. The cell slurry or cell lysate may be resuspended and shaken to redissolve prior to use in the methods described herein.

In some embodiments, the methods described herein produce a composition comprising at least 2 g/L of gamma-cyclodextrin. In some embodiments, the methods produce a composition comprising at least 3 g/L of gamma-cyclodextrin, at least 4 g/L of gamma-cyclodextrin, at least 5 g/L of gamma-cyclodextrin, at least 6 g/L gamma-cyclodextrin, at least 7 g/L gamma-cyclodextrin, at least 8 g/L gamma-cyclodextrin, at least 9 g/L of gamma-cyclodextrin, at least 10 g/L of gamma-cyclodextrin, at least 12 g/L gamma-cyclodextrin, at least 15 g/L gamma-cyclodextrin, at least 20 g/L gamma-cyclodextrin, at least 30 g/L gamma-cyclodextrin, at least 40 g/L gamma-cyclodextrin, at least 50 g/L gamma-cyclodextrin, or at least 60 g/L gamma-cyclodextrin. In a preferred embodiment, the method produces a composition comprising at least 10 g/L of gamma-cyclodextrin.

In some embodiments, the percentage yield of gamma-cyclodextrin is at least about 10%, for example at least about 20%, for example at least about 30%, for example at least about 40%, for example at least about 50%, or for example at least about 60%, wherein the percentage yield is calculated by dividing the total amount of gamma-cyclodextrin produced in the methods described herein by the maximum theoretical amount of gamma-cyclodextrin which could be produced from the starting sucrose reagent.

Also provided herein are compositions comprising cyclodextrin, wherein the cyclodextrin comprises gamma-cyclodextrin and may optionally further comprise alpha-cyclodextrin, beta-cyclodextrin, or any combination thereof, and wherein the composition comprising cyclodextrin comprises gamma-cyclodextrin in an amount and/or concentration greater than alpha-cyclodextrin, beta-cyclodextrin, or both. Preferably, the compositions are obtained from the methods provided herein. The compositions may comprise ratios of gamma-cyclodextrin to alpha-cyclodextrin, beta-cyclodextrin, or both, of at least 2:1, at least 3:1, at least 4:1, at least 5:1, at least 6:1, at least 7:1, at least 8:1, at least 9:1, at least 10:1, at least 20:1, at least 30:1, at least 40:1, at least 50:1, at least 60:1, at least 70:1, at least 80:1, at least 90:1, at least 100:1, or greater.

In a preferred embodiment, the present invention provides a method of producing a composition comprising cyclodextrin, the method comprising: (a) contacting sucrose with an enzyme or an enzyme mixture capable of converting sucrose to amylose under conditions that permit the conversion of sucrose to amylose, thereby producing amylose; (b) contacting the amylose produced in (a) with cyclodextrin glucanotransferase, thereby producing the composition comprising cyclodextrin, wherein the cyclodextrin glucanotransferase in (b) is a variant enzyme capable of producing a greater amount and/or concentration of gamma-cyclodextrin than alpha-cyclodextrin, beta-cyclodextrin, or both, relative to the wild-type enzyme capable of converting amylose to cyclodextrin, wherein the composition comprising cyclodextrin comprises gamma-cyclodextrin, and may optionally further comprise alpha-cyclodextrin, beta-cyclodextrin, or any combination thereof, wherein the ratio of gamma-cyclodextrin to alpha-cyclodextrin, beta-cyclodextrin, or both in the composition is as least 5:1, wherein steps (a) and (b) are carried out simultaneously, wherein steps (a) and (b) are carried out at from about 45° C. to about 55° C., wherein steps (a) and (b) are carried out at a pH of from about 7.0 to about 7.5, wherein steps (a) and (b) are carried out in a reaction mixture comprising water and an organic solvent (preferably toluene), and wherein the total reaction is carried out for no more than 8 hours.

Also provided herein is gamma-cyclodextrin. Preferably, the gamma-cyclodextrin is obtained from the methods provided herein.

Also provided herein is the use of sucrose as a starting material for the manufacture of gamma-cyclodextrin. Also provided herein is the use of sucrose in a method for producing gamma-cyclodextrin, wherein the method does not use starch.

Also provided herein is the use of any one of the enzyme, or enzyme mixtures, capable of converting sucrose to amylose described herein for converting sucrose into amylose.

Also provided herein is the use of any one of the enzymes capable of converting amylose to cyclodextrin described herein for converting amylose to cyclodextrin and/or for producing a greater amount and/or concentration of gamma-cyclodextrin than alpha-cyclodextrin, beta-cyclodextrin, or both.

Also provided herein is the use of any one of the enzymes, or enzyme mixtures, described herein for the manufacture of gamma-cyclodextrin, wherein the manufacture does not require starch as a starting material.

Also provided herein is any one of the enzymes, or enzyme mixtures, described herein. For example, provided herein is an enzyme comprising or consisting of an amino acid sequence of any one of SEQ ID NOs: 1-31. Also provided herein is an enzyme comprising or consisting of an amino acid sequence having at least about 70% sequence identity, preferably at least about 90% sequence identity, to the amino acid sequence of any one of SEQ ID NOs: 1-31.

Preferably, the enzyme is a variant amylosucrase enzyme comprising or consisting of an amino acid sequence of any one of SEQ ID NOs: 3-16 or 31. Also provided herein is an enzyme comprising or consisting of an amino acid sequence having at least about 70% sequence identity, preferably at least about 90% sequence identity, to the amino acid sequence of any one of SEQ ID NOs: 3-16 or 31.

Preferably the enzyme is a variant sucrose phosphorylase enzyme comprising or consisting of an amino acid sequence of SEQ ID NO: 20. Also provided herein is an enzyme comprising or consisting of an amino acid sequence having at least about 70% sequence identity, preferably at least about 90% sequence identity, to the amino acid sequence of SEQ ID NO: 20.

Preferably the enzyme is a variant alpha-glucan phosphorylase enzyme comprising or consisting of an amino acid sequence of SEQ ID NO: 24. Also provided herein is an enzyme comprising or consisting of an amino acid sequence having at least about 70% sequence identity, preferably at least about 90% sequence identity, to the amino acid sequence of SEQ ID NO: 24.

Preferably the enzyme is a variant cyclodextrin glucanotransferase enzyme comprising or consisting of an amino acid sequence of any one of SEQ ID NOs: 27-30. Also provided herein is an enzyme comprising or consisting of an amino acid sequence having at least about 70% sequence identity, preferably at least about 90% sequence identity, to the amino acid sequence of any one of SEQ ID NOs: 27-30.

Also provided herein is an enzyme composition comprising one or more of the enzymes described herein.

Also provided herein is a gene encoding any one of the variant enzymes described herein. Also provided herein is a vector encoding any one of the variant enzymes described herein. Also provided herein is a recombinant host cell comprising any one of the genes, vectors, or enzymes described herein.

Also provided herein is the use of an organic solvent, preferably toluene, for increasing the yield of gamma-cyclodextrin obtained in a method for producing gamma-cyclodextrin, such as the gamma-cyclodextrin obtained from any one of the methods described herein.

In general, the term “sequence identity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their percent identity. The percent identity of two sequences, whether nucleic acid or amino acid sequences, is the number of exact matches between two aligned sequences divided by the length of the longer sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA, 87:2264-2268 (1990) and as discussed in Altschul, et al., J. Mol. Biol., 215:403-410 (1990); Karlin And Altschul, Proc. Natl. Acad. Sci. USA, 90:5873-5877 (1993); and Altschul et al., Nucleic Acids Res., 25:3389-3402 (1997). The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 70% to 100% and integer values therebetween. In general, this disclosure encompasses sequences with at least about 70%, at least about 75%, at least about 80%, at least about 85%, at least about 90%, at least about 95%, or at least about 98% sequence identity with any sequence provided herein.

The term “about,” as used herein, generally refers to a range that is 15% greater than or less than a stated numerical value within the context of the particular usage. For example, “about 10” would include a range from 8.5 to 11.5.

As used herein, the term “or” is used nonexclusively to encompass “or” and “and.” For example, “A or B” includes “A but not B,” “B but not A,” and “A and B” unless otherwise indicated.

“A”, “an”, and “the”, as used herein, can include plural referents unless expressly and unequivocally limited to one referent.

The section headings used herein are for organizational purposes only and are not to be construed as limiting the subject matter described.

### NUMBERED EMBODIMENTS

The following embodiments recite nonlimiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1: A method of producing a composition comprising cyclodextrin, the method comprising: (a) contacting sucrose with an enzyme, or an enzyme mixture, capable of converting sucrose to amylose under conditions that permit the conversion of the sucrose to amylose, thereby producing amylose; (b) contacting the amylose produced in (a) with an enzyme capable of converting amylose to cyclodextrin under conditions that permit the conversion of the amylose to cyclodextrin, thereby producing the composition comprising cyclodextrin, wherein the composition comprising cyclodextrin comprises gamma-cyclodextrin, and may optionally further comprise alpha-cyclodextrin, beta-cyclodextrin, or any combination thereof, and wherein the composition comprising cyclodextrin comprises gamma-cyclodextrin in an amount and/or concentration greater than alpha-cyclodextrin, beta-cyclodextrin, or both.

Embodiment 2: The method of embodiment 1, wherein the enzyme of (a) is, or the enzyme mixture of (a) comprises, amylosucrase.

Embodiment 3: The method of embodiment 2, wherein the amylosucrase is a variant amylosucrase comprising at least one amino acid variant relative to a wild-type amylosucrase.

Embodiment 4: The method of embodiment 3, wherein the variant amylosucrase is capable of producing a greater amount and/or concentration of amylose from sucrose relative to a wild-type amylosucrase.

Embodiment 5: The method of embodiment 3 or 4, wherein the wild-type amylosucrase is Cellulomonas carboniz T26 amylosucrase.

Embodiment 6: The method of embodiment 5, wherein the wild-type amylosucrase comprises or consists of the amino acid sequence of SEQ ID NO: 1.

Embodiment 7: The method of embodiment 3 or 4, wherein the wild-type amylosucrase is Neisseria polysaccharea amylosucrase.

Embodiment 8: The method of embodiment 7, wherein the wild-type amylosucrase comprises or consists of the amino acid sequence of SEQ ID NO: 2.

Embodiment 9: The method of any one of embodiments 3-8, wherein the variant amylosucrase comprises or consists of an amino acid sequence having at least about 70% sequence identity to the amino acid sequence of SEQ ID NO: 1 or SEQ ID NO: 2.

Embodiment 10: The method of any one of embodiments 3-9, wherein the at least one amino acid variant comprises at least one amino acid substitution relative to a wild-type amylosucrase.

Embodiment 11: The method of embodiment 10, wherein the at least one amino acid substitution comprises an amino acid substitution at amino acid position 234 relative to a wild-type amylosucrase having the amino acid sequence of SEQ ID NO: 2.

Embodiment 12: The method of embodiment 11, wherein the amino acid substitution at position 234 is selected from the group consisting of: R234Q, R234G, R234A, R234S, R234M, R234C, R234K, R234I, R234D, R234Y, R234W, R234E, R234L, and R234H.

Embodiment 13: The method of embodiment 1, wherein the enzyme mixture of (a) comprises at least two enzymes which, collectively or in combination, are capable of converting sucrose to amylose.

Embodiment 14: The method of embodiment 13, wherein the enzyme mixture comprises sucrose phosphorylase.

Embodiment 15: The method of embodiment 14, wherein the sucrose phosphorylase is capable of converting sucrose to glucose-1-phosphate.

Embodiment 16: The method of embodiment 15, wherein the contacting of (a) further comprises contacting the sucrose with the sucrose phosphorylase under conditions that permit the conversion of the sucrose to glucose-1-phosphate.

Embodiment 17: The method of any one of embodiments 14-16, wherein the sucrose phosphorylase is selected from the group consisting of: Bifidobacterium longum sucrose phosphorylase, Leuconostoc mesenteroides sucrose phosphorylase, and Streptococcus mutans sucrose phosphorylase.

Embodiment 18: The method of any one of embodiments 14-17, wherein the sucrose phosphorylase comprises or consists of the amino acid sequence of any one of SEQ ID NOS: 17-20, or an amino acid sequence having at least about 70% sequence identity to the amino acid sequence of any one of SEQ ID NOs: 17-20.

Embodiment 19: The method of one of embodiments 13-18, wherein the enzyme mixture comprises alpha-glucan phosphorylase.

Embodiment 20: The method of embodiment 19, wherein the alpha-glucan phosphorylase is capable of converting the glucose-1-phosphate to amylose.

Embodiment 21: The method of embodiment 20, wherein the contacting of (a) further comprises contacting the glucose-1-phosphate with the alpha-glucan phosphorylase under conditions that permit the conversion of the glucose-1-phosphate to amylose.

Embodiment 22: The method of any one of embodiments 19-21, wherein the alpha-glucan phosphorylase is selected from the group consisting of: Solanum tuberosum alpha-glucan phosphorylase, S. tokodaii strain 7 alpha-glucan phosphorylase, and C. callunae DSM 20145 alpha-glucan phosphorylase.

Embodiment 23: The method of any one of embodiments 19-22, wherein the alpha-glucan phosphorylase comprises or consists of the amino acid sequence of any one of SEQ ID NOS: 21-24 or an amino acid sequence having at least about 70% sequence identity to the amino acid sequence of any one of SEQ ID NOS: 21-24.

Embodiment 24: The method of any one of embodiments 1-23, wherein the enzyme capable of converting the amylose to cyclodextrin in (b) comprises an enzyme capable of producing a greater amount and/or concentration of gamma-cyclodextrin relative to alpha-cyclodextrin, beta-cyclodextrin, or both.

Embodiment 25: The method of any one of embodiments 1-24, wherein the enzyme capable of converting the amylose to cyclodextrin in (b) is cyclodextrin glucanotransferase.

Embodiment 26: The method of embodiment 25, wherein the cyclodextrin glucanotransferase is Bacillus clarkii cyclodextrin glucanotransferase.

Embodiment 27: The method of embodiment 25 or 26, wherein the cyclodextrin glucanotransferase comprises or consists of the amino acid sequence of SEQ ID NO: 25 or 26 or an amino acid sequence having at least about 70% sequence identity to the amino acid sequence of SEQ ID NO: 25 or 26.

Embodiment 28: The method of any one of embodiments 1-27, wherein the enzyme capable of converting amylose to cyclodextrin is a variant cyclodextrin glucanotransferase.

Embodiment 29: The method of embodiment 28, wherein the variant cyclodextrin glucanotransferase is capable of producing a greater amount and/or concentration of gamma-cyclodextrin than alpha-cyclodextrin, beta-cyclodextrin, or both, relative to a wild-type cyclodextrin glucanotransferase.

Embodiment 30: The method of embodiment 29, wherein the variant cyclodextrin glucanotransferase comprises at least one amino acid variant relative to the wild-type cyclodextrin glucanotransferase.

Embodiment 31: The method of embodiment 30, wherein the wild-type cyclodextrin glucanotransferase is Bacillus clarkii cyclodextrin glucanotransferase.

Embodiment 32: The method of any one of embodiments 29-31, wherein the wild-type cyclodextrin glucanotransferase comprises or consists of the amino acid sequence of SEQ ID NOS: 25 or 26.

Embodiment 33: The method of any one of embodiments 28-32, wherein the variant cyclodextrin glucanotransferase comprises or consists of an amino acid sequence having at least about 70% sequence identity to the amino acid sequence of SEQ ID NOS: 25 or 26.

Embodiment 34: The method of any one of embodiments 30-33, wherein the at least one amino acid variant comprises at least one amino acid substitution.

Embodiment 35: The method of embodiment 34, wherein the at least one amino acid substitution comprises an amino acid substitution at amino acid position 186 relative to a wild-type cyclodextrin glucanotransferase having the amino acid sequence of SEQ ID NO: 26.

Embodiment 36: The method of embodiment 35, wherein the amino acid substitution at position 186 is Y186W.

Embodiment 37: The method of any one of embodiments 34-36, wherein the at least one amino acid substitution comprises an amino acid substitution at amino acid position 223 relative to a wild-type cyclodextrin glucanotransferase having the amino acid sequence of SEQ ID NO: 26.

Embodiment 38: The method of embodiment 37, wherein the amino acid substitution at position 223 is selected from the group consisting of: A223H, A223K, and A223R.

Embodiment 39: The method of any one of embodiments 1-38, wherein the contacting of (a) and the contacting of (b) occur sequentially.

Embodiment 40: The method of any one of embodiments 1-38, wherein the contacting of (a) and the contacting of (b) occur simultaneously or substantially simultaneously.

Embodiment 41: The method of any one of embodiments 1-40, wherein the amylose produced in (a) is not purified or isolated prior to the contacting of (b).

Embodiment 42: The method of any one of embodiments 1-41, wherein the contacting of (a), the contacting of (b), or both, is performed in vitro.

Embodiment 43: The method of embodiment 42, wherein the contacting of (a), the contacting of (b), or both, is performed in a container, a vial, a jar, a test tube, a well, a plate, or an encapsulation.

Embodiment 44: The method of embodiment 42 or 43, wherein the enzyme or at least one enzyme of the enzyme mixture of (a), the variant enzyme of (b), or both, are purified enzymes, isolated enzymes, or both.

Embodiment 45: The method of any one of embodiments 42-44, wherein the enzyme or at least one enzyme of the enzyme mixture of (a), the variant enzyme of (b), or both, are recombinantly produced enzymes.

Embodiment 46: The method of any one of embodiments 1-41, wherein the contacting of (a), the contacting of (b), or both, is performed in vivo.

Embodiment 47: The method of embodiment 46, wherein the contacting of (a), the contacting of (b), or both, is performed in a recombinant host cell.

Embodiment 48: The method of embodiment 47, wherein the recombinant host cell comprises a heterologous nucleic acid encoding the enzyme or at least one enzyme of the enzyme mixture of (a), the variant enzyme of (b), or both.

Embodiment 49: The method of embodiment 47 or 48, wherein the recombinant host cell is a microbial cell.

Embodiment 50: The method of embodiment 49, wherein the microbial cell is a bacterial cell.

Embodiment 51: The method of any one of embodiments 1-50, wherein a ratio of gamma-cyclodextrin to alpha-cyclodextrin in the composition comprising cyclodextrin is at least 2:1.

Embodiment 52: The method of any one of embodiments 1-51, wherein a ratio of gamma-cyclodextrin to beta-cyclodextrin in the composition comprising cyclodextrin is at least 2:1.

Embodiment 53: The method of any one of embodiments 1-52, wherein the composition comprising cyclodextrin comprises no or substantially no alpha-cyclodextrin, beta-cyclodextrin, or both.

### EXAMPLES

### Example 1. Variant Cyclodextrin Glucanotransferase is Capable of Increasing the Production of Gamma-Cyclodextrin Relative to Alpha-Cyclodextrin and Beta-Cyclodextrin

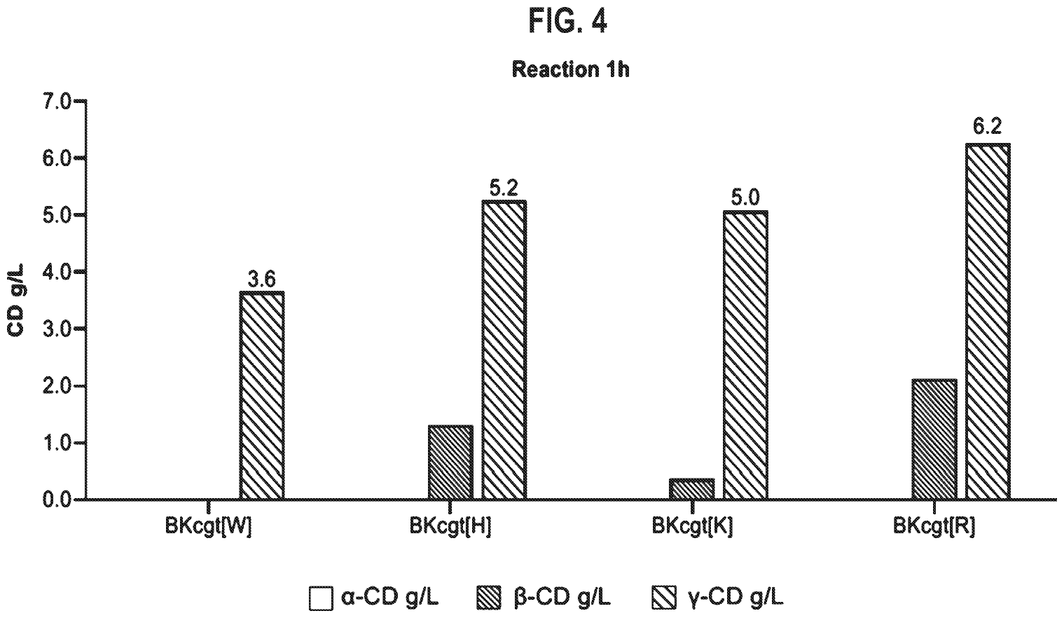

This example demonstrates that mutant cyclodextrin glucanotransferase enzymes were capable of increasing the production of gamma-cyclodextrin from amylose, relative to either alpha-cyclodextrin, beta-cyclodextrin, or both. In this example, several different cyclodextrin glucanotransferase enzymes (“BKcgt[W]” having an amino acid sequence according to SEQ ID NO: 27; “BKcgt[H]” having an amino acid sequence according to SEQ ID NO: 28, “BKcgt[K]” having an amino acid sequence according to SEQ ID NO: 29; and “BKcgt[R]” having an amino acid sequence according to SEQ ID NO: 30) were expressed in Escherichia coli and then separated from the insoluble cell debris mixture by centrifugation. The cyclodextrin glucanotransferase enzymes were exposed to soluble starch (60 g/L) for 1 hour at 55° C. in 0.1 M citric acid-sodium salt buffer at pH 8.0. The reaction was quenched using formic acid and the amounts of alpha-cyclodextrin, beta-cyclodextrin, and gamma-cyclodextrin were measured by HPLC. FIG. 4 and Table 6 below demonstrates that all tested cyclodextrin glucanotransferase enzymes (BKcgt[W], BKcgt[H], BKcgt[K], and BKcgt[R]) were capable of producing greater ratios of gamma-cyclodextrin to beta-cyclodextrin. No alpha-cyclodextrin was detected in any of the reactions.

TABLE 6
Summary of ratios of alpha-cyclodextrin to beta-cyclodextrin
and gamma-cyclodextrin generated using various cyclodextrin
glucanotransferase enzymes.

| | 1 h Ratio % | | |
|---|---|---|---|
| | α | β | γ |
| BKcgt[W] | 0 | 0.0 | 100.0 |
| BKcgt[H] | 0 | 20.0 | 80.0 |
| BKcgt[K] | 0 | 6.3 | 93.7 |
| BKcgt[R] | 0 | 25.2 | 74.8 |

### Example 2. One-Pot Synthesis of Gamma-Cyclodextrin from Sucrose

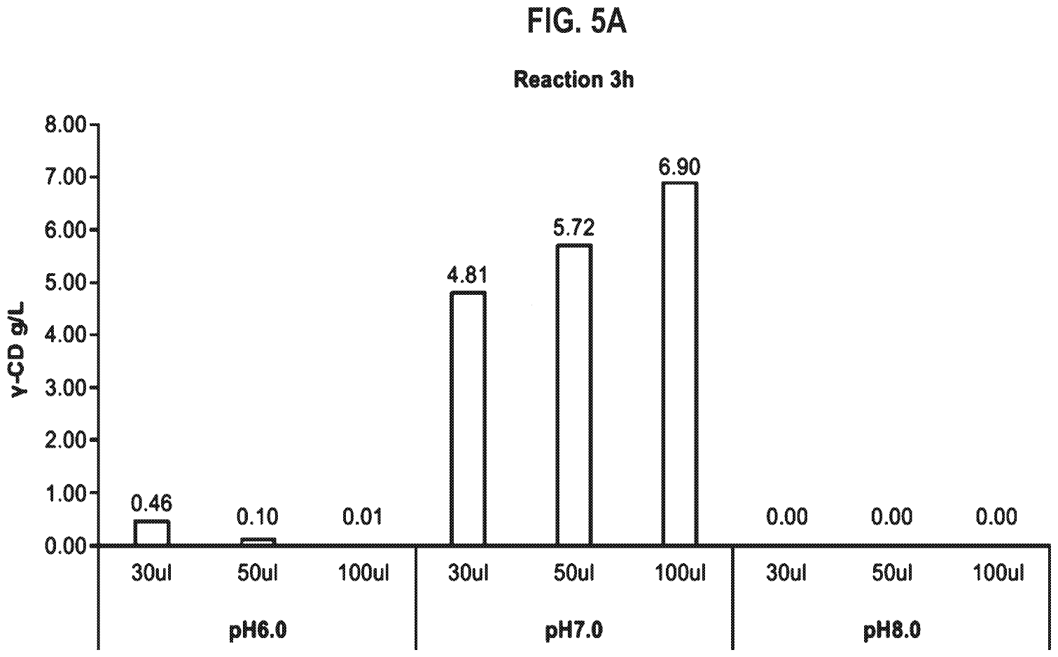

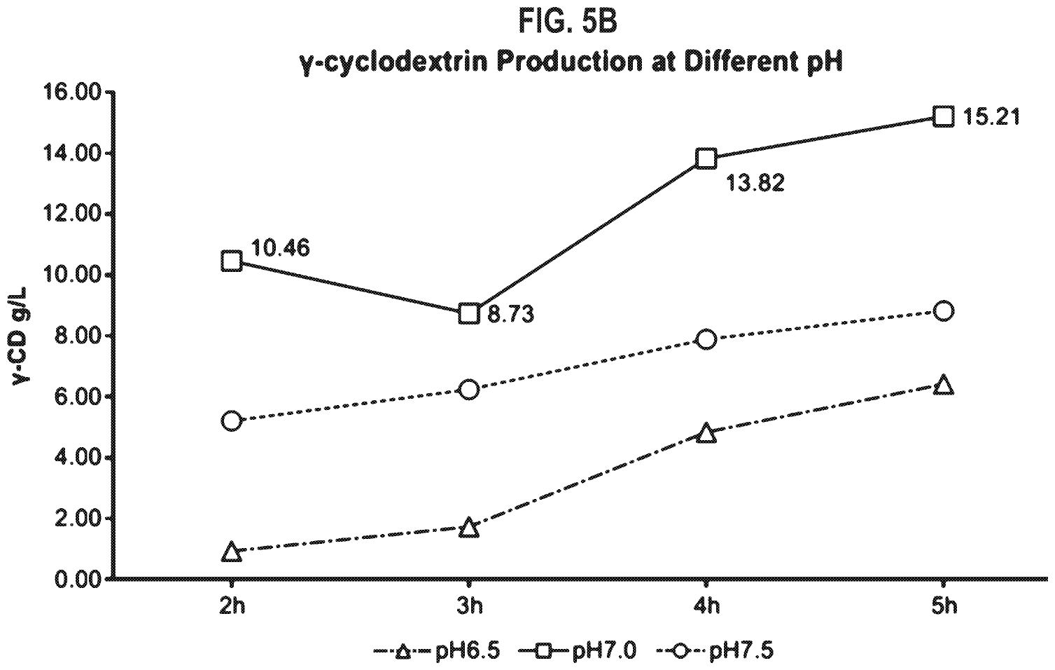

In this example, a two-enzyme system was used to produce gamma-cyclodextrin from sucrose (i.e., method step (a) was a one enzyme method (e.g., as described herein) and method step (b) was a one enzyme method (e.g., as described herein)). Amylosucrase R234Q (having an amino acid sequence according to SEQ ID NO: 3) and cyclodextrin glucanotransferase (“BKcgt[R]” having an amino acid sequence according to SEQ ID NO: 30) were expressed in Escherichia coli and then separated from the insoluble cell debris mixture by centrifugation. 200 μL of amylosucrase (SEQ ID NO: 3) and various amounts of cyclodextrin glucanotransferase (SEQ ID NO: 30; 30 μL, 50 μL, and 100 μL) was then exposed to 250 g/L sucrose at different pH (pH 6.0, pH 7.0, and pH 8.0) at 50° C. in 0.1 M citric acid-sodium salt buffer. Levels of gamma-cyclodextrin were measured by HPLC at 3 hours, as shown in FIG. 5A. The reaction was further pH optimized by using smaller pH increments between pH 6.5 and pH 7.5. This showed that the reaction worked best at pH 7.0 yet is capable of producing reasonable amounts of product at both pH 6.5 and pH 7.5. The results appear to be independent of vessel size and work best at times longer than 1 hour and at pH around 7.0, as shown in FIG. 5B.

FIGS. 5A and 5B demonstrate that one-pot synthesis reactions are capable of producing gamma-cyclodextrin from sucrose under various different pH conditions. These reactions use sucrose as a starting point and rely on the two-enzyme system composed of Amylosucrase R234Q (having an amino acid sequence according to SEQ ID NO: 3) and cyclodextrin glucanotransferase (“BKcgt[R]” having an amino acid sequence according to SEQ ID NO: 30).

### Example 3. Freeze-Drying Amylosucrase Cell Slurry and Cell Lysates with Additives

Amylosucrase (having an amino acid sequence according to SEQ ID NO: 3) lysates and whole cell slurry were freeze-dried with different additives. In more detail, 0.5%, 1.0%, or 5.0% w/v of PEG, maltose, sorbitol, sucrose, glucose, mannitol, lactose, milk powder, starch or beta-cyclodextrin were added to 1 mL of the lysate or cell slurry. The mixtures were then freeze-dried over two days.

The resultant material was resuspended in 1 mL of water and shaken at 1200 rpm for 30 minutes at room temperature (about 25° C.) to allow for dissolution. The retained enzymatic activity of the resultant solutions was measured as described below.

### Amylosucrase Enzymatic Activity

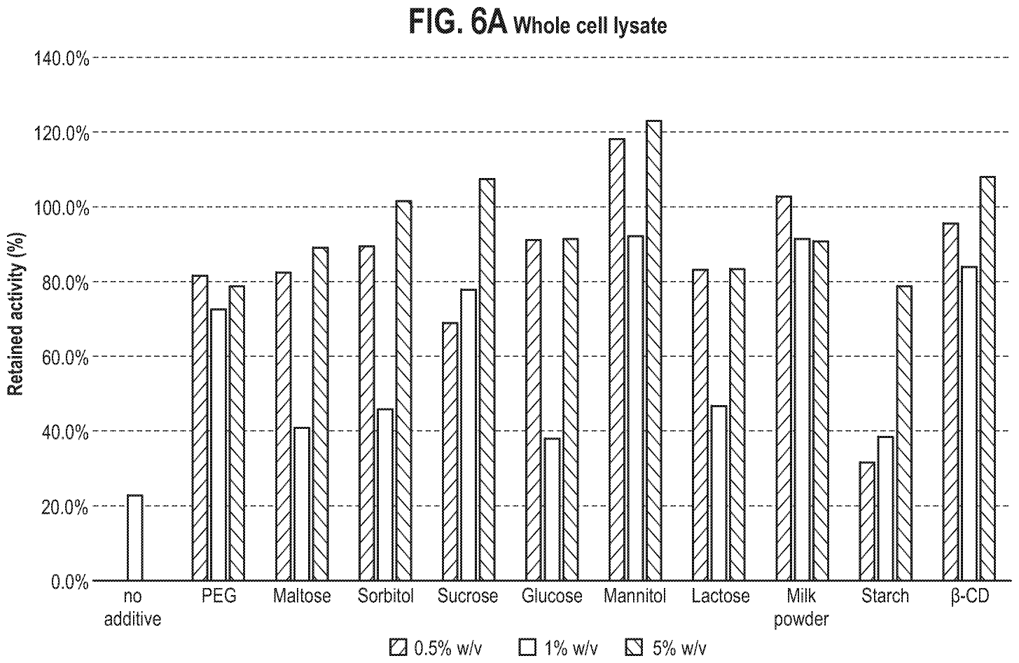

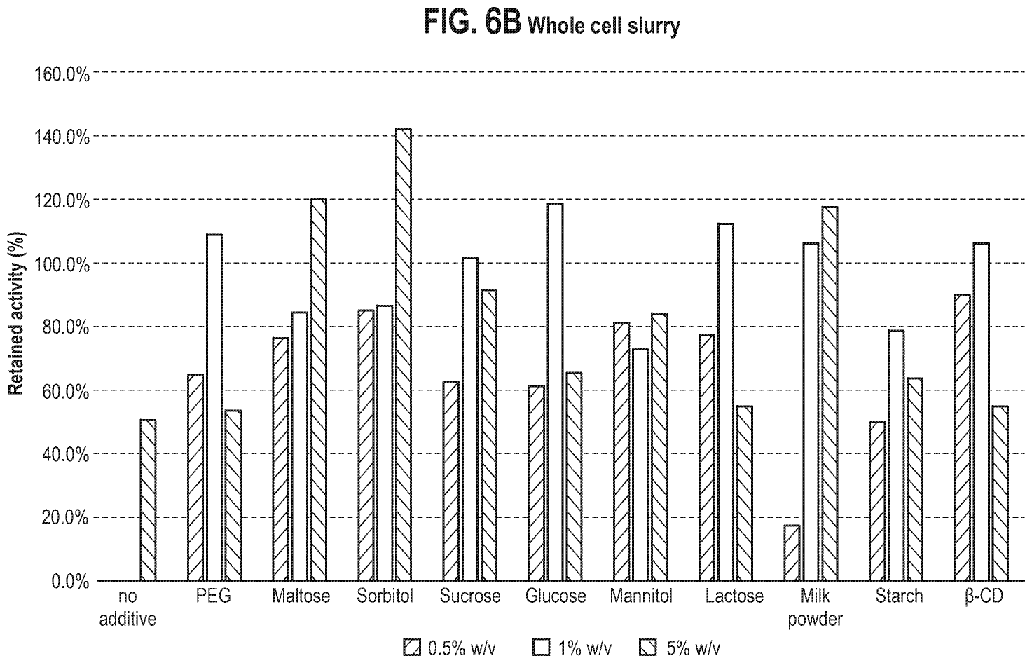

33 μL of the amylosucrase cell free lysate (or whole cell slurry) was added to a 2.67 mL solution of sucrose (100 g/L) in Na citrate buffer (0.1 M), at pH 7 and 40° C. The solution was shaken at 1200 rpm for 1 hour. The concentration of starch was quantified by spectrophotometric analysis. One unit of activity (U/mL) was defined as the amount of enzyme required to produce 1 g/L amylose per minute at pH 7, and at 40° C. The activity was compared to cell lysate (or whole cell slurry respectively) that had not been freeze-dried, and the results are shown in FIGS. 6A and 6B respectively.

As shown in FIG. 6, the addition of additives to amylosucrase prior to freeze-drying improves the stability of the enzymes. In particular, addition of 5% sucrose, 0.5% mannitol, and 0.5% sorbitol demonstrated an improved retained enzyme activity for amylosucrase cell lysate and whole cell slurry.

The examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of producing a composition comprising cyclodextrin, the method comprising:
(a) contacting sucrose with an enzyme, or an enzyme mixture, capable of converting sucrose to amylose under conditions that permit the conversion of the sucrose to amylose, thereby producing amylose; and
(b) contacting the amylose produced in (a) with an enzyme capable of converting amylose to cyclodextrin under conditions that permit the conversion of the amylose to cyclodextrin, thereby producing the composition comprising cyclodextrin,
wherein the composition comprising cyclodextrin comprises gamma-cyclodextrin in an amount and/or concentration greater than alpha-cyclodextrin, beta-cyclodextrin, or both.

2. The method of claim 1, wherein the enzyme of (a) is, or the enzyme mixture of (a) comprises, amylosucrase and/or sucrose phosphorylase.

3. The method of claim 2, wherein the amylosucrase is a variant amylosucrase comprising at least one amino acid variant relative to a wild-type amylosucrase.

4. The method of claim 3, wherein the wild-type amylosucrase is:
(i) Cellulomonas carboniz T26 amylosucrase; or
(ii) Neisseria polysaccharea amylosucrase.

5. The method of claim 3, wherein the variant amylosucrase comprises or consists of an amino acid sequence having at least about 70% sequence identity to the amino acid sequence of SEQ ID NO: 1 or SEQ ID NO: 2.

6. The method of claim 1, wherein the at least one amino acid variant comprises at least one amino acid substitution relative to a wild-type amylosucrase,
wherein the at least one amino acid substitution comprises an amino acid substitution at amino acid position 234 relative to a wild-type amylosucrase having the amino acid sequence of SEQ ID NO: 2.

7. The method of claim 1, wherein the enzyme mixture of (a) comprises at least two enzymes, and wherein the enzyme mixture comprises sucrose phosphorylase.

8. (canceled)

9. The method of claim 7, wherein the sucrose phosphorylase is selected from the group consisting of: Bifidobacterium longum sucrose phosphorylase, Leuconostoc mesenteroides sucrose phosphorylase, and Streptococcus mutans sucrose phosphorylase.

10. The method of claim 7, wherein the sucrose phosphorylase comprises or consists of the amino acid sequence of any one of SEQ ID NOS: 17-20, or an amino acid sequence having at least about 70% sequence identity, to the amino acid sequence of any one of SEQ ID NOs: 17-20.

11. (canceled)

12. The method of claim 7, wherein the sucrose phosphorylase is alpha-glucan phosphorylase and A) the alpha-glucan phosphorylase is selected from the group consisting of: Solanum tuberosum alpha-glucan phosphorylase, S. tokodaii strain 7 alpha-glucan phosphorylase, and C. callunae DSM 20145 alpha-glucan phosphorylase and/or B) the alpha-glucan phosphorylase comprises or consists of the amino acid sequence of any one of SEQ ID NOS: 21-24 or an amino acid sequence having at least about 70% sequence identity to the amino acid sequence of any one of SEQ ID NOS: 21-24.

13.-15. (canceled)

16. The method of claim 1, wherein the enzyme capable of converting the amylose to cyclodextrin in (b) is cyclodextrin glucanotransferase and wherein the cyclodextrin glucanotransferase is Bacillus clarkii cyclodextrin glucanotransferase, and/or
wherein the cyclodextrin glucanotransferase comprises or consists of the amino acid sequence of SEQ ID NO: 25 or 26 or an amino acid sequence having at least about 70% sequence identity to the amino acid sequence of SEQ ID NO: 25 or 26.

17. The method of claim 1, wherein the enzyme capable of converting amylose to cyclodextrin in (b) comprises a variant cyclodextrin glucanotransferase.

18. (canceled)

19. The method of claim 17, wherein the variant cyclodextrin glucanotransferase comprises at least one amino acid variant relative to the wild-type cyclodextrin glucanotransferase,
wherein the wild-type cyclodextrin glucanotransferase is Bacillus clarkii cyclodextrin glucanotransferase, and/or
wherein the variant cyclodextrin glucanotransferase comprises or consists of an amino acid sequence having at least about 70% sequence identity to the amino acid sequence of SEQ ID NOS: 25 or 26.

20. (canceled)

21. The method of claim 19, wherein the at least one amino acid substitution comprises an amino acid substitution at amino acid position 186 relative to a wild-type cyclodextrin glucanotransferase having the amino acid sequence of SEQ ID NO: 26.

22. The method of claim 19, wherein the at least one amino acid substitution comprises an amino acid substitution at amino acid position 223 relative to a wild-type cyclodextrin glucanotransferase having the amino acid sequence of SEQ ID NO: 26.

23. (canceled)

24. The method of claim 1, wherein the amylose produced in (a) is not purified or isolated prior to the contacting of (b).

25.-29. (canceled)

30. The method of claim 1, wherein a ratio of gamma-cyclodextrin to alpha-cyclodextrin and/or beta-cyclodextrin in the composition comprising cyclodextrin is at least 2:1.

31.-36. (canceled)

37. The method of claim 36, wherein the enzyme or at least one enzyme of the enzyme mixture of (a), the variant enzyme of (b), or both, is provided in a cell slurry or whole cell lysate, and wherein the cell slurry or whole cell lysate further comprises an additive selected from the group consisting of
mannitol, sorbitol, sucrose and combinations thereof.

38. The method of claim 1, wherein steps (a) and (b) are carried out at a pH of from about 7.0 to about 7.5.

39. A composition comprising cyclodextrin, wherein the cyclodextrin comprises gamma-cyclodextrin and may optionally further comprise alpha-cyclodextrin, beta-cyclodextrin, or any combination thereof, and wherein the composition comprising cyclodextrin comprises gamma-cyclodextrin in an amount and/or concentration greater than alpha-cyclodextrin, beta-cyclodextrin, or both, and wherein the composition is obtained from the method of claim 1.

40.-43. (canceled)

44. An enzyme comprising or consisting of an amino acid sequence of any one of SEQ ID NOs: 1-31, or an enzyme comprising or consisting of an amino acid sequence having at least about 70% sequence identity to the amino acid sequence of any one of SEQ ID NOs: 1-31.

45. (canceled)

46. The method of claim 6, wherein the amino acid substitution at position 234 is R234Q.

47. The method of claim 1, wherein the enzyme or enzyme mixture capable of converting sucrose to amylose under conditions that permit the conversion of the sucrose to amylose of (a) is or comprises an amino acid sequence of SEQ ID NO: 3.

48. The method of claim 1, wherein the enzyme capable of converting amylose to cyclodextrin of (b) is or comprises an amino acid sequence of SEQ ID NO: 30.


SEQUENCE LISTING (31 sequences)

SEQ ID NO: 1 - protein, 644 aa, Cellulomonas carbonis
  source 1..644: note = Cellulomonas carbonis T26
MRVSEPLHPSVTAAVAAARDPRTSAEHWAAIEARVAREWPRLERLFLEVYGEGERTRSELAALAHQLVVSAQERPADLRAVDVAREADPAWFTSHRMLGGVCYVDRYAGDLEGLRRHIPYLRELGLTYLHLMPLFEAPAENSDGGYAVSSYRRVNPALGTMEQLTALAAELRENGISLVLDFIFNHTSDEHEWARRALAGEREYEDYYWVFPDREMPDAYERTVREIFPDDHPGSFVPMPDSPDGSRTGRWIWATFHSFQWDLNYANPAVFRAMAGEMLFLANKGVDVLRMDAVAFIWKQLGTACESLPQAHLLIQAFNAALRIAAPGVLFKSEAIVHPDEVVQYISPDECQISYNPLQMALIWSSLATREANLLQQALERRHALPPGTAWVNYVRSHDDIGWTFADEDAAELGIDGFQHRRFLNAFYVDRFPGSFARGVPFQDNPRTGDCRISGTTASLAGLEARDPGAVDRILLAHSIVLSTGGIPLLYLGDEVGQLNDYSYRDQPGLAEDSRWVNRPWYPAQAYANRLVPSTAAGRVFRGLRHLLEVRRRTPELAGGTLVPFDAHNRHVVGYQRPGELDGVPTTVLCLASFADEPQAVEPLTLSGMPAEAEDLLTGATVDLRAGLVLRPHGFVWLRVRHAA

SEQ ID NO: 2 - protein, 636 aa, Neisseria polysaccharea
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLREIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 3 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLQEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 4 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLGEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 5 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLAEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 6 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLSEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 7 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLMEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 8 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLCEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 9 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLKEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 10 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLIEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 11 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLDEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 12 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLYEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 13 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLWEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 14 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLEEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 15 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLLEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 16 - protein, 636 aa, synthetic construct
MLTPTQQVGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLHEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 17 - protein, 508 aa, Bifidobacterium longum
MKNKVQLITYADRLGDGTLSSMADILRTRFDGVYDGVHILPFFTPFDGADAGFDPIDHTKVDERLGSWDDVAELSKTHNIMVDAIVNHMSWESKQFQDVLEKGEESEYYPMFLTMSSVFPNGATEEDLAGIYRPRPGLPFTHYKFAGKTRLVWVSFTPQQVDIDTDSDKGWEYLMSIFDQMAASHVSYIRLDAVGYGAKEAGTSCFMTPKTFKLISRLREEGVKRGLEILIEVHSYYKKQVEIASKVDRVYDFALPPLLLHSLFTGHVEPVAHWTEIRPNNAVTVLDTHDGIGVIDIGSDQLDRSLKGLVPDEDVDNLVNTIHANTHGESQAATGAAASNLDLYQVNSTYYSALGCNDQHYLAARAVQFFLPGVPQVYYVGALAGRNDMELLRRTNNGRDINRHYYSTAEIDENLERPVVKALNALAKFRNELPAFDGEFSYEVDGDTSITFRWTAADGTSTAALTFEPGRGLGTDNATPVASLAWSDAAGDHETRDLLANPPIADID

SEQ ID NO: 18 - protein, 490 aa, Leuconostoc mesenteroides
MEIQNKAMLITYADSLGKNLKDVHQVLKEDIGDAIGGVHLLPFFPSTGDRGFAPADYTRVDAAFGDWADVEALGEEYYLMFDFMINHISRESVMYQDFKKNHDDSKYKDFFIRWEKFWAKAGENRPTQADVDLIYKRKDKAPTQEITFDDGTTENLWNTFGEEQIDIDVNSAIAKEFIKTTLEDMVKHGANLIRLDAFAYAVKKVDTNDFFVEPEIWDTLNEVREILTPLKAEILPEIHEHYSIPKKINDHGYFTYDFALPMTTLYTLYSGKTNQLAKWLKMSPMKQFTTLDTHDGIGVVDARDILTDDEIDYASEQLYKVGANVKKTYSSASYNNLDIYQINSTYYSALGNDDAAYLLSRVFQVFAPGIPQIYYVGLLAGENDIALLESTKEGRNINRHYYTREEVKSEVKRPVVANLLKLLSWRNESPAFDLAGSITVDTPTDTTIVVTRQDENGQNKAVLTADAANKTFEIVENGQTVMSSDNLTQN

SEQ ID NO: 19 - protein, 481 aa, Streptococcus mutans
MPIINKTMLITYADSLGKNLKELNENIENYFGDAVGGVHLLPFFPSTGDRGFAPIDYHEVDSAFGDWDDVKCLGEKYYLMFDFMINHISRQSKYYKDYQEKHEASAYKDLFLNWDKFWPKNRPTQEDVDLIYKRKDRAPKQEIQFADGSVEHLWNTFGEEQIDLDVTKEVTMDFIRSTIENLAANGCDLIRLDAFAYAVKKLDTNDFFVEPEIWTLLDKVRDIAAVSGAEILPEIHEHYTIQFKIADHDYYVYDFALPMVTLYSLYSSKVDRLAKWLKMSPMKQFTTLDTHDGIGVVDVKDILTDEEITYTSNELYKVGANVNRKYSTAEYNNLDIYQINSTYYSALGDDDQKYFLARLIQAFAPGIPQVYYVGFLAGKNDLELLESTKEGRNINRHYYSSEEIAKEVKRPVVKALLNLFTYRNQSAAFDLDGRIEVETPNEATIVIERQNKDGSHIAKAEINLQDMTYRVTENDQTISFE

SEQ ID NO: 20 - protein, 481 aa, synthetic construct
MPITNKTMLITYADSLGKNLKELNENIENYFGDAVGGVHLLPFFPSSGDRGFAPIDYHEVDPAFGDWDDVKRLGEKHYLMFDFMINHISRQSKYYKDYQEKHEASAYKDLFLNWDKFWPKNRPTQEDLDLIYKRKDRAPMQEIRFADGSVEHLWSTFGEEQIDLDVTKEVTMDFIRSTIENLAANGCDLIRLDAFAYAVKKLDTNDFFVEPEIWTLLDKVRDIAAVSGAEILPEIHEHYTIQFKIADHGYYVYDFALPMVTLYSLYSGKVDRLAKWLKMSPMKQFTTLDTHDGIGVVDVKDILTDEEITYTSNELYKVGANVNRKYSTAEYNNLDIYQINSTYYSALGDDDQKYFLARLIQAFAPGIPQVYYVGFLAGKNDLELLESTKEGRNINRHYYSSEEIAKEVKRPVVKALLNLFTYRNQSAAFDLDGRIEVETPNEATIVIERQNKDGSHIATAEINLQDMTYRVTENDQTISFE

SEQ ID NO: 21 - protein, 916 aa, Solanum tuberosum
TLSEKIHHPITEQGGESDLSSFAPDAASITSSIKYHAEFTPVFSPERFELPKAFFATAQSVRDSLLINWNATYDIYEKLNMKQAYYLSMEFLQGRALLNAIGNLELTGAFAEALKNLGHNLENVASQEPDAALGNGGLGRLASCFLDSLATLNYPAWGYGLRYKYGLFKQRITKDGQEEVAEDWLEIGSPWEVVRNDVSYPIKFYGKVSTGSDGKRYWIGGEDIKAVAYDVPIPGYKTRTTISLRLWSTQVPSADFDLSAFNAGEHTKACEAQANAEKICYILYPGDESEEGKILRLKQQYTLCSASLQDIISRFERRSGDRIKWEEFPEKVAVQMNDTHPTLCIPELMRILIDLKGLNWNEAWNITQRTVAYTNHTVLPEALEKWSYELMQKLLPRHVEIIEAIDEELVHEIVLKYGSMDLNKLEEKLTTMRILENFDLPSSVAELFIKPEISVDDDTETVEVHDKVEASDKVVTNDEDDTGKKTSVKIEAAAEKDIDKKTPVSPEPAVIPPKKVRMANLCVVGGHAVNGVAEIHSEIVKEEVFNDFYELWPEKFQNKTNGVTPRRWIRFCNPPLSAIITKWTGTEDWVLKTEKLAELQKFADNEDLQNEWREAKRSNKIKVVSFLKEKTGYSVVPDAMFDIQVKRIHEYKRQLLNIFGIVYRYKKMKEMTAAERKTNFVPRVCIFGGKAFATYVQAKRIVKFITDVGATINHDPEIGDLLKVVFVPDYNVSVAELLIPASDLSEHISTAGMEASGTSNMKFAMNGCIQIGTLDGANVEIREEVGEENFFLFGAQAHEIAGLRKERADGKFVPDERFEEVKEFVRSGAFGSYNYDDLIGSLEGNEGFGRADYFLVGKDFPSYIECQEKVDEAYRDQKRWTTMSILNTAGSYKFSSDRTIHEYAKDIWNIEAVEIA

SEQ ID NO: 22 - protein, 480 aa, Sulfurisphaera tokodaii
MRKHLKGKAHKKLHMIISITAELGIDFGENFAGGLGVLEGDKFYASARLGIDYTVFTLFYRKGYTGNEEKQKELLKNLVKEWETEIELKKGKIKIEYLTYKLNTAKAIFINILSPDWAKRLNEKLYIENSEEDRFYKYLVLAKATEKYISEKIGWDKIKYVDLQEAYPSFLPLLKYFPRYRIIIHTPAPWGHPTFPARYFKEEFGFEFPFDPVVMTEIGLSSAVQGIVVSKKMLHHVSKTFPHHMHKIKAITNAVEIPRWRHPLLNNVKDLDDFIKKKKEVKKESLKKLGKESDKPTIGWVRRITQYKRPEFILRLIDELRDDVVFIIGGKAHPYEYYGVELEKKFKEYAQKRNNVIYVQGVDIQQMKLAIWSSDIWTFTPYSGWEASGTSFMKAGVNGVPSVASRDGAVPEIIKDGYNGWLYGEDRYELLPVDTYDREYEEFARKVKEALNKYYEVGYNAYHTFSDFCSMDRLMKEYAL

SEQ ID NO: 23 - protein, 796 aa, Corynebacterium callunae
MSPEKQPLPAALVGSHVRAAAGTPADLATDRKFWTGLSRAVQERIADDWERTREAYGAARQQHYFSAEFLMGRALLNNLTNLGLVDEAAAATRELGHELTDILEIENDAALGNGGLGRLAACFLDSAVTQDYPVTGYGLLYRFGLFRQSFNEGFQVEKPDPWREEEYPFTIRRASDQLVVCFDDMKTRAIPYDMPITGYGTHNVGTLRLWKAEPWEEFDYDAFNSQRFTDAIIERERVSDICRVLYPNDTTYEGKKLRVRQQYFFTSASLQAMIQDHLAHHKDLSNFAEFHSVQLNDTHPVLAIPELMRLLMDEHDMGWEESWAIVSKTFAYTNHTVLTEALEQWDEQIFQQLFWRVWEIIAEIDRRFRLERAADGLDEETINRMAPIQHGTVHMAWIACYAAYSINGVAALHTEIIKAETLADWYALWPEKFNNKTNGVTPRRWLRMINPGLSDLLTRLSGSDDWVTDLDELKKLRSYADDKSVLEELRAIKAANKQDFAEWILERQGIEIDPESIFDVQIKRLHEYKRQLMNALYVLDLYFRIKEDGLTDIPARTVIFGAKAAPGYVRAKAIIKLINSIADLVNNDPEVSPLLKVVFVENYNVSPAEHILPASDVSEQISTAGKEASGTSNMKFMMNGALTLGTMDGANVEIVDSVGEENAYIFGARVEELPALRESYKPYELYETVPGLKRALDALDNGTLNDNNSGLFYDLKHSLIHGYGKDASDTYYVLGDFADYRETRDRMAADYASDPLGWARMAWINICESGRFSSDRTIRDYATEIWKLEPTPAVKK

SEQ ID NO: 24 - protein, 916 aa, synthetic construct
TLSEKIHHPITEQGGESDLSSFAPDAASITSSIKYHAELTPVFSPERFELPKAFFATAQSVRDSLLINWNATYDIYEKLNMKQAYYLSMEFLQGRALLNAIGNLELTGAFAEALKNLGHNLENVASQEPDAALGSGGLGRLASCFLDSLATLNYPAWGYGLRYKYGLFKQRITKDGQEEVAEDWLEIGSPWEVVRNDVSYPIKFYGKVSTGSDGKRYWIGGEDIKAVAYDVPIPGYKTRTTISLRLWSTQVPSADFDLSAFNAGEHTKACEAQANAEKICYILYPGDESEEGKILRLKQQYTLCSASLQDIISRFERRSGDRIKWEEFPEKVAVQMNDTHPTLCIPELMRILIDLKGLNWNEAWNITQRTVAYTNHTVLPEALEKWSYELMQKLLPRHVEIIEAIDEELVHEIVLKYGSMDLNKLEEKLTTMRILENFDLPSSVAELFIKPEISVDDDTETVEVHDKVEASDKVVTNDEDDTGKKTSVKIEAAAEKDIDKKTPVSPEPAVIPPKKVRMANLCVVGGHAVNGVAEIHSEIVKEEVFNDFYELWPEKFQNKTNGVTPRRWIRFCNPPLSAIITKWTGTEDWVLKTEKLAELQKFADNEDLQNEWREAKRSNKIKVVSFLKEKTGYSVVPDAMFDIQVKRIHEYKRQLLNIFGIVYRYKKMKEMTAAERKTNFVPRVCIFGGKAFATYVQAKRIVKFIIDVGATINHDPEIGDLLKVVFVPDYNVSVAELLIPASDLSEHISTAGMEASGTSNMKFAMNGCIQIGTLDGANVEIREEVGEENFFLFGAQAHEIAGLRKERADGKFVPDERFEEVKEFVRSGAFGSYNYDDLIGSLEGNEGFGRADYFLVGKDFPSYIECQEKVDEAYRDQKRWTTMSILNTAGSYKFSSDRTIHEYAKDIWNIEAVEIA

SEQ ID NO: 25 - protein, 702 aa, Bacillus clarkii
MFRKLLCTLVTIITLSAWIVSHGGEVHASNATNDLSNVNYAEEVIYHIVTDRFKDGDPDNNPQGQLFSNGCSDLTKYCGGDWQGIIDEIESGYLPDMGITALWISPPVENVFDLHPEGFSSYHGYWARDFKKTNPFFGDFDDFSRLIETAHAHDIKVVIDFVPNHTSPVDIEDGALYDNGTLLGHYSTDANNYFYNYGGSDFSDYENSIYRNLYDLASLNQQHSFIDKYLKESIQLWLDTGIDGIRVDAVAHMPLGWQKAFISSVYDYNPVFTFGEWFTGAQGSNHYHHFVNNSGMSALDFRYAQVAQDVLRNQKGTMHDIYDMLASTQLDYERPQDQVTFIDNHDIDRFTVEGRDTRTTDIGLAFLLTSRGVPAIYYGTENYMTGKGDPGNRKMMESFDQTTTAYQVIQKLAPLRQENKAVAYGSTKERWINDDVLIYERSFNGDYLLVAINKNVNQAYTISGLLTEMPAQVYHDVLDSLLDGQSLAVKENGTVDSFLLGPGEVSVWQHISESGSAPVIGQVGPPMGKPGDAVKISGSGFGSEPGTVYFRDTKIDVLTWDDETIVITLPETLGGKAQISVTNSDGVTSNGYDFQLLTGKQESVRFVVDNAHTNYGENVYLVGNVPELGNWNPADAIGPMFNQVVYSYPTWYYDVSVPADTALEFKFIIVDGNGNVTWESGGNHNYRVTSGSTDTVRVSFRR

SEQ ID NO: 26 - protein, 674 aa, Bacillus clarkii
SNATNDLSNVNYAEEVIYHIVTDRFKDGDPDNNPQGQLFSNGCSDLTKYCGGDWQGIIDEIESGYLPDMGITALWISPPVENVFDLHPEGFSSYHGYWARDFKKTNPFFGDFDDFSRLIETAHAHDIKVVIDFVPNHTSPVDIEDGALYDNGTLLGHYSTDANNYFYNYGGSDFSDYENSIYRNLYDLASLNQQHSFIDKYLKESIQLWLDTGIDGIRVDAVAHMPLGWQKAFISSVYDYNPVFTFGEWFTGAQGSNHYHHFVNNSGMSALDFRYAQVAQDVLRNQKGTMHDIYDMLASTQLDYERPQDQVTFIDNHDIDRFTVEGRDTRTTDIGLAFLLTSRGVPAIYYGTENYMTGKGDPGNRKMMESFDQTTTAYQVIQKLAPLRQENKAVAYGSTKERWINDDVLIYERSFNGDYLLVAINKNVNQAYTISGLLTEMPAQVYHDVLDSLLDGQSLAVKENGTVDSFLLGPGEVSVWQHISESGSAPVIGQVGPPMGKPGDAVKISGSGFGSEPGTVYFRDTKIDVLTWDDETIVITLPETLGGKAQISVTNSDGVTSNGYDFQLLTGKQESVRFVVDNAHTNYGENVYLVGNVPELGNWNPADAIGPMFNQVVYSYPTWYYDVSVPADTALEFKFIIVDGNGNVTWESGGNHNYRVTSGSTDTVRVSFRR

SEQ ID NO: 27 - protein, 674 aa, synthetic construct
SNATNDLSNVNYAEEVIYHIVTDRFKDGDPDNNPQGQLFSNGCSDLTKYCGGDWQGIIDEIESGYLPDMGITALWISPPVENVFDLHPEGFSSYHGYWARDFKKTNPFFGDFDDFSRLIETAHAHDIKVVIDFVPNHTSPVDIEDGALYDNGTLLGHYSTDANNYFYNYGGSDFSDYENSIYRNLWDLASLNQQHSFIDKYLKESIQLWLDTGIDGIRVDAVAHMPLGWQKAFISSVYDYNPVFTFGEWFTGAQGSNHYHHFVNNSGMSALDFRYAQVAQDVLRNQKGTMHDIYDMLASTQLDYERPQDQVTFIDNHDIDRFTVEGRDTRTTDIGLAFLLTSRGVPAIYYGTENYMTGKGDPGNRKMMESFDQTTTAYQVIQKLAPLRQENKAVAYGSTKERWINDDVLIYERSFNGDYLLVAINKNVNQAYTISGLLTEMPAQVYHDVLDSLLDGQSLAVKENGTVDSFLLGPGEVSVWQHISESGSAPVIGQVGPPMGKPGDAVKISGSGFGSEPGTVYFRDTKIDVLTWDDETIVITLPETLGGKAQISVTNSDGVTSNGYDFQLLTGKQESVRFVVDNAHTNYGENVYLVGNVPELGNWNPADAIGPMFNQVVYSYPTWYYDVSVPADTALEFKFIIVDGNGNVTWESGGNHNYRVTSGSTDTVRVSFRR

SEQ ID NO: 28 - protein, 674 aa, synthetic construct
SNATNDLSNVNYAEEVIYHIVTDRFKDGDPDNNPQGQLFSNGCSDLTKYCGGDWQGIIDEIESGYLPDMGITALWISPPVENVFDLHPEGFSSYHGYWARDFKKTNPFFGDFDDFSRLIETAHAHDIKVVIDFVPNHTSPVDIEDGALYDNGTLLGHYSTDANNYFYNYGGSDFSDYENSIYRNLYDLASLNQQHSFIDKYLKESIQLWLDTGIDGIRVDAVHHMPLGWQKAFISSVYDYNPVFTFGEWFTGAQGSNHYHHFVNNSGMSALDFRYAQVAQDVLRNQKGTMHDIYDMLASTQLDYERPQDQVTFIDNHDIDRFTVEGRDTRTTDIGLAFLLTSRGVPAIYYGTENYMTGKGDPGNRKMMESFDQTTTAYQVIQKLAPLRQENKAVAYGSTKERWINDDVLIYERSFNGDYLLVAINKNVNQAYTISGLLTEMPAQVYHDVLDSLLDGQSLAVKENGTVDSFLLGPGEVSVWQHISESGSAPVIGQVGPPMGKPGDAVKISGSGFGSEPGTVYFRDTKIDVLTWDDETIVITLPETLGGKAQISVTNSDGVTSNGYDFQLLTGKQESVRFVVDNAHTNYGENVYLVGNVPELGNWNPADAIGPMFNQVVYSYPTWYYDVSVPADTALEFKFIIVDGNGNVTWESGGNHNYRVTSGSTDTVRVSFRR

SEQ ID NO: 29 - protein, 674 aa, synthetic construct
SNATNDLSNVNYAEEVIYHIVTDRFKDGDPDNNPQGQLFSNGCSDLTKYCGGDWQGIIDEIESGYLPDMGITALWISPPVENVFDLHPEGFSSYHGYWARDFKKTNPFFGDFDDFSRLIETAHAHDIKVVIDFVPNHTSPVDIEDGALYDNGTLLGHYSTDANNYFYNYGGSDFSDYENSIYRNLYDLASLNQQHSFIDKYLKESIQLWLDTGIDGIRVDAVKHMPLGWQKAFISSVYDYNPVFTFGEWFTGAQGSNHYHHFVNNSGMSALDFRYAQVAQDVLRNQKGTMHDIYDMLASTQLDYERPQDQVTFIDNHDIDRFTVEGRDTRTTDIGLAFLLTSRGVPAIYYGTENYMTGKGDPGNRKMMESFDQTTTAYQVIQKLAPLRQENKAVAYGSTKERWINDDVLIYERSFNGDYLLVAINKNVNQAYTISGLLTEMPAQVYHDVLDSLLDGQSLAVKENGTVDSFLLGPGEVSVWQHISESGSAPVIGQVGPPMGKPGDAVKISGSGFGSEPGTVYFRDTKIDVLTWDDETIVITLPETLGGKAQISVTNSDGVTSNGYDFQLLTGKQESVRFVVDNAHTNYGENVYLVGNVPELGNWNPADAIGPMFNQVVYSYPTWYYDVSVPADTALEFKFIIVDGNGNVTWESGGNHNYRVTSGSTDTVRVSFRR

SEQ ID NO: 30 - protein, 674 aa, synthetic construct
SNATNDLSNVNYAEEVIYHIVTDRFKDGDPDNNPQGQLFSNGCSDLTKYCGGDWQGIIDEIESGYLPDMGITALWISPPVENVFDLHPEGFSSYHGYWARDFKKTNPFFGDFDDFSRLIETAHAHDIKVVIDFVPNHTSPVDIEDGALYDNGTLLGHYSTDANNYFYNYGGSDFSDYENSIYRNLYDLASLNQQHSFIDKYLKESIQLWLDTGIDGIRVDAVRHMPLGWQKAFISSVYDYNPVFTFGEWFTGAQGSNHYHHFVNNSGMSALDFRYAQVAQDVLRNQKGTMHDIYDMLASTQLDYERPQDQVTFIDNHDIDRFTVEGRDTRTTDIGLAFLLTSRGVPAIYYGTENYMTGKGDPGNRKMMESFDQTTTAYQVIQKLAPLRQENKAVAYGSTKERWINDDVLIYERSFNGDYLLVAINKNVNQAYTISGLLTEMPAQVYHDVLDSLLDGQSLAVKENGTVDSFLLGPGEVSVWQHISESGSAPVIGQVGPPMGKPGDAVKISGSGFGSEPGTVYFRDTKIDVLTWDDETIVITLPETLGGKAQISVTNSDGVTSNGYDFQLLTGKQESVRFVVDNAHTNYGENVYLVGNVPELGNWNPADAIGPMFNQVVYSYPTWYYDVSVPADTALEFKFIIVDGNGNVTWESGGNHNYRVTSGSTDTVRVSFRR

SEQ ID NO: 31 - protein, 629 aa, synthetic construct
MGLILQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLNEIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQGLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA